(19) United States
(12) Patent Application Publication
(10) Pub. No.: US 2012/0135521 A1
(43) Pub. Date: May 31, 2012

(54) ANTIGENE LOCKS AND THERAPEUTIC USES THEREOF

(76) Inventors: James R. Eshleman, Lutherville, MD (US); Antony R. Parker, Eastbourne (GB)

(21) Appl. No.: 13/247,366

(22) Filed: Sep. 28, 2011

Related U.S. Application Data
(63) Continuation of application No. 10/505,308, filed on Sep. 7, 2005.
(60) Provisional application No. 60/359,116, filed on Feb. 22, 2002.
(60) Provisional application No. 60/359,614, filed on Feb. 25, 2002.
(60) Provisional application No. 60/366,674, filed on Mar. 22, 2002.

(51) Int. Cl. C12N 5/02; C07H 21/04

(52) U.S. Cl. 435/375; 536/24.5

(57) ABSTRACT

An oligonucleotide based therapeutic strategy, called anti-gene locks, is described which specifically kills cells based on their genotype. The strategy employs oligonucleotides with arms and a backbone that are complementary to both strands of the gene target. Anti-gene locks bind in vitro in a sequence dependent fashion and inhibit DNA synthesis. In bacterial cells containing an episome target, they cause elimination of the extra-chromosomal DNA structure. When the target is present in the bacterial or human genome, they selectively kill the majority of these cells.

The present application claims the benefit of U.S. provisional application No. 60/359,116 filed Feb. 22, 2002, U.S. provisional application No. 60/359,614 filed Feb. 25, 2002 and U.S. provisional application 60/366,674 filed Mar. 22, 2002 which are incorporated by reference, herein, in their entirety.

### BACKGROUND OF THE INVENTION

1. Field of the Invention

Antigene locks bind in a sequence dependent manner to their target genes in-vitro and inhibit in-vitro DNA synthesis. When transformed into cells, they cause elimination or degradation of a non-essential extra-chromosomal genetic element (F′ episome). Moreover, anti-gene locks can specifically and selectively kill either bacterial or human cells if the target is present in their genomes.

2. Background

In 1994, Nilsson and colleagues described an in situ hybridization technique, designated “padlock probes”, which can detect single base mutations yet be seen at the light microscope level (Nilsson, M. et al. “Padlock probes: circularizing oligonucleotides for localized DNA detection”. Science 265, 2085-8 (1994). Padlock probes are large oligonucleotides, whose arms are complementary to, and wrap around the target DNA in an end-to-end orientation, and are then ligated if a perfect match exists between the arms and target. Since both arms are typically about twenty bases each, together they are expected to wrap around a DNA target approximately four times before being locked through ligation (one turn per ˜10 bases). In this way they are inextricably bound to the target (hence “padlock”), permitting highly stringent washing prior to detection, using either the biotin molecules in the non-complementary backbone or through rolling circle amplification.

With many diseases, patients exist as cell chimeras, in that they have acquired a second cell population (e.g. malignant cells, bacterial cells, HIV infected cells). In each case, this second cell population contains an additional gene or genes, which not only define these cells as unique, but also could be used to target this second cell population in the treatment of a patient.

While existing approaches to target cells based on their genotype is limited, some molecular based approaches have been developed. These include antisense RNA [(Izant, J. G. & Weintraub, H. Science 229, 345-52. (1985); Detrick, B. et al. Invest. Ophthalmol. Vis. Sci. 42, 163-9. (2001); Miller, P. S., Cassidy, R. A., Hamma, T. & Kondo, N. S. Pharmacol. Ther. 85, 159-63. (2000)], triplex DNA [(Blume, S. W., Gee, J. E., Shrestha, K. & Miller, D. M. Nucleic Acids Res 20, 1777-84. (1992); Chan, P. P. & Glazer, P. M. J. Mol. Med. 75, 267-82. (1997); Cassidy, R. A., Kondo, N. S. & Miller, P. S. Biochemistry 39, 8683-91. (2000)], ribozymes [(Beaudry, A. A. & Joyce, G. F. Science 257, 635-41. (1992); Joyce, G. F. Science 289, 401-2. (2000)], “suicide” gene therapy [(Shimura, H. et al. Cancer Res. 61, 3640-6. (2001); Black, M. E., Kokoris, M. S. & Sabo, P. Cancer Res. 61, 3022-6. (2001)], and inhibitory RNA [(Elbashir, S. M. et al. Nature 411, 494-8 (2001); Brummelkamp, T. R., Bernards, R. & Agami, R. Science 296, 550-3 (2002)]. However, each of these approaches has its limitations, e.g. the triplex DNA approach is somewhat limited by the need to target homopurine/homopyrimidine tracts exclusively. Moreover, most of these technologies target messenger RNA rather than the unique genes directly.

There is a need in the art to selectively target foreign genetic material, whether integrated or present as an extra-chromosomal episome, to inhibit nucleic acid synthesis and resulting in selective cell death.

### SUMMARY OF THE INVENTION

Sequence specific antigene locks bind to a target nucleic acid molecule, inhibiting the expression thereof. Antigene locks are effective in the treatment of abnormal cell growth and diseases caused by infectious disease agents.

In particular, the invention provides methods for inhibiting replication or transcription of a nucleic acid molecule indicative of a disease state, comprising:

targeting the nucleic acid molecule with an oligonucleotide; and,

binding of the oligonucleotide to the target nucleic acid molecule.

In a preferred embodiment, the oligonucleotide comprises a backbone nucleic acid sequence with two arms. Preferably, the backbone and arms are complementary to a target nucleic acid molecule. In addition the nucleic acid sequences of the arms are preferably, complementary to the backbone nucleic acid sequences.

In another preferred embodiment, the antigene oligonucleotide is comprised of one arm comprising a 5′ to 3′ nucleic acid sequence which is complementary to a 3′ to 5′ nucleic acid sequence comprising the backbone. The other arm is a 3′ to 5′ nucleic acid sequence and is complementary to a 5′ to 3′ nucleic acid sequence comprising the backbone.

In one aspect the 5′ to 3′ arm and the 3′ to 5′ arm comprise an equal ratio of nucleic acid bases. In another aspect the 5′ to 3′ arm and the 3′ to 5′ arm comprise a varying ratio of nucleic acid bases so that one arm comprises a larger number of nucleobases as compared to the other arm. For example, the ratio of nucleic acid bases of the 5′ to 3′ arm and the 3′ to 5′ arm vary between about 0.1:1 to about 20:1.

In one preferred embodiment, the backbone comprises at least one mismatching base compared to the arm having a complementary nucleic acid sequence. The backbone also comprises at least one mismatching base compared to the target nucleic acid molecule it is designed to target.

In another aspect of the invention, the antigene oligonucleotide hybridizes with genomic target molecules as well as episomal structures. Preferably, the 5′ arm ligates to the 3′ arm after the oligonucleotide has hybridized to its target nucleic acid molecule, either genomic and/or episomal, thereby forming a locked complex. In a most preferred embodiment, the locked complex inhibits replication of the nucleic acid sequence and/or the locked complex inhibits transcription in vitro or in vivo.

In a preferred embodiment, antigene locks which have hybridized to DNA or RNA target, antigene locks can be ligated by, for example, native cellular ligases. Alternatively, the ends of the antigene locks may be chemically modified such that they self-ligate when the ends are juxtaposed on their specific target. See, for example, Sando and Kool, J. Am. Chem. Soc., 124: 9686-9687, 2002 which is incorporated herein, in its entirety. Examples of chemical modifications include, but are not limited to: dabsyl and thioate moeities.

In another preferred embodiment, the antigene locks comprise molecules or oligonucleotide sequences comprising ligase activity. For example, PCR products are cloned, using standard TA cloning, but in which a vector is designed to comprise topoisomerase recognition sequences (e.g. CCCTT), and in which topoisomerases (e.g. topoisomerase I isolated from Vaccinia), comprising ligase activity is covalently ligated to the cloning vector (Shuman et al, J. Biol. Chem., 269: 32678-32684, 1994; Heyman et al, Genome Research, 9: 383-392, 1999). Similarly, a ligase or topoisomerase or other enzyme possessing ligase activity could be covalently attached to the antigene locks to facilitate ligation after target binding.

In another preferred embodiment, the target nucleic acid molecule in a cell is expressed in a disease state or is a foreign nucleic acid molecule. The disease state is cancer and/or an infectious disease organism, such as a virus. Other infectious disease organisms include bacteria, protozoa or fungi. The bacterium can be a multi-drug resistant bacterium.

In another preferred embodiment, the antigene locks inhibit the expression of a target nucleic molecule in cells of in an organism in need of treatment. The cell with the target gene may be derived from or contained in any organism. The organism may a plant, animal, protozoan, bacterium, virus, or fungus. The plant may be a monocot, dicot or gymnosperm; the animal may be a vertebrate or invertebrate. Preferred microbes are those used in agriculture or by industry, and those that are pathogenic for plants or animals. Fungi include organisms in both the mold and yeast morphologies.

Plants include arabidopsis; field crops (e.g., alfalfa, barley, bean, corn, cotton, flax, pea, rape, rice, rye, safflower, sorghum, soybean, sunflower, tobacco, and wheat); vegetable crops (e.g., asparagus, beet, broccoli, cabbage, carrot, cauliflower, celery, cucumber, eggplant, lettuce, onion, pepper, potato, pumpkin, radish, spinach, squash, taro, tomato, and zucchini); fruit and nut crops (e.g., almond, apple, apricot, banana, blackberry, blueberry, cacao, cherry, coconut, cranberry, date, fajoa, filbert, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut, and watermelon); and ornamentals (e.g., alder, ash, aspen, azalea, birch, boxwood, camellia, carnation, chrysanthemum, elm, fir, ivy, jasmine, juniper, oak, palm, poplar, pine, redwood, rhododendron, rose, and rubber).

Examples of vertebrate animals include fish, mammal, cattle, goat, pig, sheep, rodent, hamster, mouse, rat, primate, and human; invertebrate animals include nematodes, other worms, drosophila, and other insects. Representative generae of nematodes include those that infect animals (e.g., Ancylostoma, Ascaridia, Ascaris, Bunostomum, Caenorhabditis, Capillaria, Chabertia, Cooperia, Dictyocaulus, Haemonchus, Heterakis, Nematodirus, Oesophagostomum, Ostertagia, Oxyuris, Parascaris, Strongylus, Toxascaris, Trichuris, Trichostrongylus, Tfhchonema, Toxocara, Uncinaria) and those that infect plants (e.g., Bursaphalenchus, Criconemella, Diiylenchus, Ditylenchus, Globodera, Helicotylenchus, Heterodera, Longidorus, Melodoigyne, Nacobbus, Paratylenchus, Pratylenchus, Radopholus, Rotelynchus, Tylenchus, and Xiphinema). Representative orders of insects include Coleoptera, Diptera, Lepidoptera, and Homoptera.

The cell having the target gene may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

In accordance with the invention, the antigen lock is not limited to any type of target gene or nucleotide sequence. But the following classes of possible target genes are listed for illustrative purposes: developmental genes (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogenes (e.g., ABL1, BCL1, BCL2, BCL6, CBFA2, CBL, CSF1R, ERBA, ERBB, EBRB2, ETS1, ETS1, ETV6, FGR, FOS, FYN, HCR, HRAS, JUN, KRAS, LCK, LYN, MDM2, MLL, MYB, MYC, MYCL1, MYCN, NRAS, PIM1, PML, RET, SRC, TAL1, TCL3, and YES); tumor suppressor genes (e.g., APC, BRCA1, BRCA2, MADH4, MCC, NF1, NF2, RB1, TP53, and WT1); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases).

In another preferred embodiment, the antigene oligonucleotide comprises a total of from about 8 to about 200 base units, more preferably, a total of from about 8 to about 150 base units, most preferably, the antigene oligonucleotide comprises a total of from about 10 to about 100 base units. However, different sizes of an antigene lock can be designed depending on the target nucleic acid sequence.

In one preferred embodiment, the antigene oligonucleotide comprises modified base units. Preferably, these modified bases comprise phosphorthiorate, methylphosphonate, peptide nucleic acids, and/or LNA molecules. The antigene oligonucleotide comprises either about 1, 2, 3, 4, 5, 6, 7, 8, 9 or 10 modified base units, or may be comprised entirely of modified bases.

In one aspect of the invention the antigene oligonucleotide invades a double stranded molecule in the region of the target sequence by denaturing the bonds between the complementary target sequences of the double stranded molecule.

Preferably the antigene oligonucleotide has equal or higher specificity and affinity for a target oligonucleotide sequence than the complementary target oligonucleotide sequence. In one aspect, the association constant (Kₐ) of the oligonucleotide for the target nucleic acid molecule is higher than the association constant of the complementary strands of a double stranded molecule. In another aspect, the association constant (Kₐ) of the oligonucleotide for the target nucleic acid molecule is higher than a disassociation constant (K_{d}) of the complementary strand of the target sequence in a double stranded molecule.

In another embodiment, the antigene oligonucleotide can bind to a wild type gene sequence and any alleles or variants thereof.

Preferably, the antigene oligonucleotide binds to double-stranded DNA target molecules as well as single-stranded DNA targets, messenger RNA and/or RNA secondary structures. The invention may be used against protein coding genes as well as non-protein coding genes. Examples of non-protein coding genes include genes that encode ribosomal RNAs, transfer RNAs, small nuclear RNAs, small cytoplasmic RNAs, telomerase RNA, RNA molecules involved in DNA replication, chromosomal rearrangement of, for instance immunoglobulin genes, etc. It may also include introns, or regions between genes.

In a preferred embodiment, the invention provides a method for selectively treating cells comprising an infectious disease organism, comprising:

administering to the cells an oligonucleotide sequence that is complementary to a target sequence of an infectious disease organism, or the cells comprising an oligonucleotide sequence of an infectious disease organism.

Preferably, the cells are mammalian cells and the cells are infected with a virus bacteria, protozoa or fungi. The cells do not have to be actively replicating and can be in any one of G1, S, M, or G2 stage of a cell cycle.

In one aspect of the invention the antigene oligonucleotide binds to a wild type infectious disease organisms' target gene sequence and any alleles or variants thereof. The foreign target nucleic acid molecule can be single-stranded DNA targets, double-stranded DNA target molecules as well as single or double stranded viral RNA targets, messenger RNA and/or RNA secondary structures. The antigene oligonucleotide preferably hybridizes with genomic target molecules as well as episomal structures.

In another aspect the 5′ end of the antigene oligonucleotide and the 3′ end of the antigene oligonucleotide wrap around the target nucleic acid molecule after the antigene oligonucleotide has hybridized to its target nucleic acid molecule thereby forming a helix. Preferably, the antigene oligonucleotide ligates to the 3′ end of the oligonucleotide after formation of the helix, forming a locked complex and the locked complex inhibits replication of the target nucleic acid sequence. The locked complex preferably also inhibits transcription.

As discussed supra, the antigene oligonucleotide can comprise modified base units, such as for example, at least one modified unit or a combination thereof, comprising phosphorthiorate, methylphosphonate, peptide nucleic acids, and/or LNA molecules.

In another preferred embodiment, the invention provides a method for treating a mammal suffering from or susceptible to an infectious disease or cancer, the method comprising:

administering to the mammal a therapeutically effective amount of an oligonucleotide. Preferably, the administered oligonucleotide hybridizes to the gene to inhibit expression thereof and/or results in inhibition of gene expression.

Preferably, the infectious disease is caused by or associated with a virus, bacteria, protozoa or fungi. In one aspect, the infectious agent is present in any tissue or organ of a mammal and the infectious agent is associated with undesired expression of at least a portion of a sequence identified in table 1, 2, 4, 5, or 6 and/or variants thereof.

In other preferred embodiments, the virus is HPV and the antigene oligonucleotide the HPV sequence as identified by SEQ. ID. NO 2.

In other preferred embodiment, antigene locks bind to repressor genes and inhibit the activity of repressor molecules. For example, in the treatment of myocardial disease an inotropic effect is desired. Pharmacological therapies have been directed toward increasing the force of contraction of the heart (by using inotropic agents such as digitalis and β-adrenergic receptor agonists), reducing fluid accumulation in the lungs and elsewhere (by using diuretics), and reducing the work of the heart (by using agents that decrease systemic vascular resistance such as angiotensin converting enzyme inhibitors). Antigene locks that inhibit the production of molecules which repress the activity of genes in a disease state are well-within the scope of the invention.

In another preferred embodiment, antigene locks are used in the treatment of individuals who are hypersensitive or allergic to certain allergens. Antigene locks are produced which prevent the expression of IgE molecules specific for certain allergens. Such antigene locks inhibit the rearrangement of immunoglobulin genes with a specificity for such allergens.

The invention may be used against protein coding genes as well as non-protein coding genes. Examples of non-protein coding genes include genes that encode ribosomal RNAs, transfer RNAs, small nuclear RNAs, small cytoplasmic RNAs, telomerase RNA, RNA molecules involved in DNA replication, chromosomal rearrangement of, for instance immunoglobulin genes, etc.

The following definitions are provided:

As used herein, the term “DNA repair gene” refers to a gene that is part of a DNA repair pathway, that when altered, permits mutations to occur in the DNA of the organism.

As used herein, the terms “exon” and “intron” are art-understood terms referring to various portions of genomic gene sequences. “Exons” are those portions of a genomic gene sequence that encode protein. “Introns” are sequences of nucleotides found between exons in genomic gene sequences. The antigene locks can be targeted to exons and/or to introns.

As used herein, the term “wrap around” refers to the binding of the antigene lock to the target nucleic acid sequence and forming a double helical structure similar to the structure double helix observed prior to transcription of a gene. The backbone and arms of the antigene lock are complementary to the target nucleic acid molecule and displace the complementary strands in the target nucleic acid molecule. Binding of the antigene lock backbone and arms occurs in a Watson-Crick type of base pairing, Hoogsteen or reverse Hoogsteen types of base pairing, or the like. The arms bind the target nucleic acid in an end-to-end orientation, and can then be ligated, either by native cellular ligases or chemical modifications. Once the antigene lock has bound to the target nucleic acid sequence, the strands twist around each other as is typical of a double stranded molecule thereby forming a double helix. For example, if both arms are about twenty bases each, together they are expected to wrap around a DNA target approximately four times before being locked through ligation (one turn per about 10 nucleobases). In this way they are inextricably bound to the target as determined by highly stringent washing and gel mobility assays as described in the Examples which follow. “Turns” refers to the natural twisting of nucleic acid bases which form a helical structure. Therefore, when an antigene lock “wraps around” a target nucleic acid, the backbone and arms of the antigen lock have bound to the 5′ to 3′ strand and the complementary 3′ to 5′ strand of the target sequence so that the binding of a contiguous stretch of nucleic acids in a 5′ to 3′ direction to its complementary 3′ to 5′ acid sequence is disrupted and instead the contiguous target sequences are bound to the complementary sequences of the antigene lock. The “wrapping around” is due to the unique design of the antigene lock wherein the backbone is complementary to the target sequence, (for example if a target strand is in a 5′ to 3′ direction the complementary backbone is in a 3′ to 5′ direction) and one arm is complementary to the 3′ to 5′ (of the target sequence) in a 5′ to 3′ direction and the other arm is in the opposite orientation so that the ends of the arms are juxtaposed, thereby completing the wrapping around of a target nucleic acid sequence by the antigene lock, i.e. the 3′ end terminal nucleic acid molecule of the 5′ to 3′ arm is juxtaposed to 5′ end terminal nucleic acid molecule of the 3′ to 5′ arm. Once an antigene lock has bound and wrapped around a target sequence, that target sequence cannot be transcribed, i.e. “locked.” The juxtaposed arms can be ligated by native cellular ligases and/or by chemical modifications of selected nucleic acids comprising the 5′ to 3′ and 3′ to 5′ arms of antigene lock. This is illustrated in FIG. 1.

As used herein, the singular forms “a”, “an” and “the” include plural referents unless the context clearly dictates otherwise.

As used herein, the term “infectious agent” refers to an organism wherein growth/multiplication leads to pathogenic events in humans or animals. Examples of such agents are: bacteria, fungi, protozoa and viruses.

As used herein, the term “oligonucleotide specific for” refers to an oligonucleotide having a sequence (i) capable of forming a stable complex with a portion of the targeted gene, e.g. by either strand invasion or triplex formation, a mechanism also called antigene or (ii) capable of forming a stable duplex with a portion of a mRNA transcript of the targeted gene a mechanism also called antisense.

As used herein, the terms “oligonucleotide”, “antigene” “antigene oligonucleotide” and “antigene locks” are used interchangeably throughout the specification and include linear or circular oligomers of natural and/or modified monomers or linkages, including deoxyribonucleosides, ribonucleosides, substituted and alpha-anomeric forms thereof, peptide nucleic acids (PNA), locked nucleic acids (LNA), phosphorthiorate, methylphosphonate, and the like. Oligonucleotides are capable of specifically binding to a target polynucleotide by way of a regular pattern of monomer-to-monomer interactions, such as Watson-Crick type of base pairing, Hoogsteen or reverse Hoogsteen types of base pairing, or the like.

The oligonucleotide may be composed of a single region or may be composed of several regions. For example, hinge regions comprising different lengths and base composition. The oligonucleotide may be “chimeric”, that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide in the case of an oligonucleotide compound. These oligonucleotides typically are comprised of at least one region wherein the oligonucleotide is modified in order to exhibit one or more desired properties. The desired properties of the oligonucleotide include, but are not limited, for example, to increased resistance to nuclease degradation, increased cellular uptake, and/or increased binding affinity for the target nucleic acid. Different regions of the oligonucleotide may therefore have different properties.

The chimeric oligonucleotides of the present invention can be formed as mixed structures of two or more oligonucleotides, modified oligonucleotides, oligonucleosides and/or oligonucleotide analogs as described above.

The oligonucleotide can be composed of regions that can be linked in “register”, that is, when the monomers are linked consecutively, as in native DNA, or linked via spacers. The spacers are intended to constitute a covalent “bridge” between the regions and have in preferred cases a length not exceeding about 100 carbon atoms. The spacers may carry different functionalities, for example, having positive or negative charge, carry special nucleic acid binding properties (intercalators, groove binders, toxins, fluorophors etc.), being lipophilic, inducing special secondary structures like, for example, alanine containing peptides that induce alpha-helices.

As used herein, the term “monomers” typically indicates monomers linked by phosphodiester bonds or analogs thereof to form oligonucleotides ranging in size from a few monomeric units, e.g., from about 3-4, to about several hundreds of monomeric units. Analogs of phosphodiester linkages include: phosphorothioate, phosphorodithioate, methylphosphornates, phosphoroselenoate, phosphoramidate, and the like, as more fully described below.

In the present context, the terms “nucleobase” covers naturally occurring nucleobases as well as non-naturally occurring nucleobases. It should be clear to the person skilled in the art that various nucleobases which previously have been considered “non-naturally occurring” have subsequently been found in nature. Thus, “nucleobase” includes not only the known purine and pyrimidine heterocycles, but also heterocyclic analogues and tautomers thereof. Illustrative examples of nucleobases are adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolopyridin, isocytosine, isoguanin, inosine and the “non-naturally occurring” nucleobases described in Benner et al., U.S. Pat. No. 5,432,272. The term “nucleobase” is intended to cover every and all of these examples as well as analogues and tautomers thereof. Especially interesting nucleobases are adenine, guanine, thymine, cytosine, and uracil, which are considered as the naturally occurring nucleobases in relation to therapeutic and diagnostic application in humans.

As used herein, “nucleoside” includes the natural nucleosides, including 2′-deoxy and 2′-hydroxyl forms, e.g., as described in Kornberg and Baker, DNA Replication, 2nd Ed. (Freeman, San Francisco, 1992).

“Analogs” in reference to nucleosides includes synthetic nucleosides having modified base moieties and/or modified sugar moieties, e.g., described generally by Scheit, Nucleotide Analogs, John Wiley, New York, 1980; Freier & Altmann, Nucl. Acid. Res., 1997, 25(22), 4429-4443, Toulmé, J. J., Nature Biotechnology 19:17-18 (2001); Manoharan M., Biochemica et Biophysica Acta 1489:117-139 (1999); Freier S. M., Nucleic Acid Research, 25:4429-4443 (1997), Uhlman, E., Drug Discovery & Development, 3: 203-213 (2000), Herdewin P., Antisense & Nucleic Acid Drug Dev., 10:297-310 (2000); 2′-O, 3′-C-linked [3.2.0] bicycloarabinonucleosides (see e.g. N. K Christiensen., et al, J. Am. Chem. Soc., 120: 5458-5463 (1998). Such analogs include synthetic nucleosides designed to enhance binding properties, e.g., duplex or triplex stability, specificity, or the like.

The term “stability” in reference to duplex or triplex formation generally designates how tightly an antisense oligonucleotide binds to its intended target sequence; more particularly, “stability” designates the free energy of formation of the duplex or triplex under physiological conditions. Melting temperature under a standard set of conditions, e.g., as described below, is a convenient measure of duplex and/or triplex stability. Preferably, oligonucleotides of the invention are selected that have melting temperatures of at least 45° C. when measured in 100 mM NaCl, 0.1 mM EDTA and 10 mM phosphate buffer aqueous solution, pH 7.0 at a strand concentration of both the oligonucleotide and the target nucleic acid of 1.5 μM. Thus, when used under physiological conditions, duplex or triplex formation will be substantially favored over the state in which the antigen and its target are dissociated. It is understood that a stable duplex or triplex may in some embodiments include mismatches between base pairs and/or among base triplets in the case of triplexes. Preferably, modified oligonucleotides, e.g. comprising LNA units, of the invention form perfectly matched duplexes and/or triplexes with their target nucleic acids.

As used herein, the term “downstream” when used in reference to a direction along a nucleotide sequence means in the direction from the 5′ to the 3′ end. Similarly, the term “upstream” means in the direction from the 3′ to the 5′ end.

As used herein, the term “gene” means the gene and all currently known variants thereof and any further variants which may be elucidated.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “variant,” when used in the context of a polynucleotide sequence, may encompass a polynucleotide sequence related to a wild type gene. This definition may also include, for example, “allelic”, “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type target genes. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base. The presence of SNPs may be indicative of, for example, a certain population with a propensity for a disease state, that is susceptibility versus resistance.

As used herein, the term “mRNA” means the presently known mRNA transcript(s) of a targeted gene, and any further transcripts which may be elucidated.

The term, “complementary” means that two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. Normally, the complementary sequence of the oligonucleotide has at least 80% or 90%, preferably 95%, most preferably 100%, complementarity to a defined sequence. Preferably, alleles or variants thereof can be identified. A BLAST program also can be employed to assess such sequence identity.

The term “complementary sequence” as it refers to a polynucleotide sequence, relates to the base sequence in another nucleic acid molecule by the base-pairing rules. More particularly, the term or like term refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 95% of the nucleotides of the other strand, usually at least about 98%, and more preferably from about 99% to about 100%. Complementary polynucleotide sequences can be identified by a variety of approaches including use of well-known computer algorithms and software, for example the BLAST program.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, a “pharmaceutical salt” include, but are not limited to, mineral or organic acid salts of basic residues such as amines; alkali or organic salts of acidic residues such as carboxylic acids. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The most preferred salt is the hydrochloride salt.

As used herein, “cancer” refers to all types of cancer or neoplasm or malignant tumors found in mammals, including, but not limited to: leukemias, lymphomas, melanomas, carcinomas and sarcomas. Examples of cancers are cancer of the brain, breast, pancreas, cervix, colon, head & neck, kidney, lung, non-small cell lung, melanoma, mesothelioma, ovary, sarcoma, stomach, uterus and Medulloblastoma.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic). Accordingly, the present invention includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Examples of sarcomas which can be treated with antigene locks and optionally a potentiator and/or chemotherapeutic agent include, but not limited to a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated with antigene locks and optionally a potentiator and/or another chemotherapeutic agent include but not limited to, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Carcinomas which can be treated with antigene locks and optionally a potentiator and/or a chemotherapeutic agent include but not limited to, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

Additional cancers which can be treated with antigene locks according to the invention include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

A “heterologous” component refers to a component that is introduced into or produced within a different entity from that in which it is naturally located. For example, a polynucleotide derived from one organism and introduced by genetic engineering techniques into a different organism is a heterologous polynucleotide which, if expressed, can encode a heterologous polypeptide. Similarly, a promoter or enhancer that is removed from its native coding sequence and operably linked to a different coding sequence is a heterologous promoter or enhancer.

A “promoter,” as used herein, refers to a polynucleotide sequence that controls transcription of a gene or coding sequence to which it is operably linked. A large number of promoters, including constitutive, inducible and repressible promoters, from a variety of different sources, are well known in the art and are available as or within cloned polynucleotide sequences (from, e.g., depositories such as the ATCC as well as other commercial or individual sources).

An “enhancer,” as used herein, refers to a polynucleotide sequence that enhances transcription of a gene or coding sequence to which it is operably linked. A large number of enhancers, from a variety of different sources are well known in the art and available as or within cloned polynucleotide sequences (from, e.g., depositories such as the ATCC as well as other commercial or individual sources). A number of polynucleotides comprising promoter sequences (such as the commonly-used CMV promoter) also comprise enhancer sequences.

“Operably linked” refers to a juxtaposition, wherein the components so described are in a relationship permitting them to function in their intended manner. A promoter is operably linked to a coding sequence if the promoter controls transcription of the coding sequence. Although an operably linked promoter is generally located upstream of the coding sequence, it is not necessarily contiguous with it. An enhancer is operably linked to a coding sequence if the enhancer increases transcription of the coding sequence. Operably linked enhancers can be located upstream, within or downstream of coding sequences. A polyadenylation sequence is operably linked to a coding sequence if it is located at the downstream end of the coding sequence such that transcription proceeds through the coding sequence into the polyadenylation sequence.

A “replicon” refers to a polynucleotide comprising an origin of replication which allows for replication of the polynucleotide in an appropriate host cell. Examples include replicons of a target cell into which a heterologous nucleic acid might be integrated (e.g., nuclear and mitochondrial chromosomes), as well as extrachromosomal replicons (such as replicating plasmids and episomes).

“Gene delivery,” “gene transfer,” and the like as used herein, are terms referring to the introduction of an exogenous polynucleotide (sometimes referred to as a “transgenes”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

“In vivo” gene delivery, gene transfer, gene therapy and the like as used herein, are terms referring to the introduction of a vector comprising an exogenous polynucleotide directly into the body of an organism, such as a human or non-human mammal, whereby the exogenous polynucleotide is introduced to a cell of such organism in vivo.

A cell is “transduced” by a nucleic acid when the nucleic acid is translocated into the cell from the extracellular environment. Any method of transferring a nucleic acid into the cell may be used; the term, unless otherwise indicated, does not imply any particular method of delivering a nucleic acid into a cell. A cell is “transformed” by a nucleic acid when the nucleic acid is transduced into the cell and stably replicated. A vector includes a nucleic acid (ordinarily RNA or DNA) to be expressed by the cell. A vector optionally includes materials to aid in achieving entry of the nucleic acid into the cell, such as a viral particle, liposome, protein coating or the like. A “cell transduction vector” is a vector which encodes a nucleic acid capable of stable replication and expression in a cell once the nucleic acid is transduced into the cell.

As used herein, a “target cell” or “recipient cell” refers to an individual cell or cell which is desired to be, or has been, a recipient of exogenous nucleic acid molecules, polynucleotides and/or proteins. The term is also intended to include progeny of a single cell.

A “vector” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a coding sequence of interest in gene therapy. Vectors include, for example, viral vectors (such as adenoviruses (“Ad”), adeno-associated viruses (AAV), and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities) mediating binding and uptake), or vectors can be modified to provide such functionalities. Other vectors include those described by Chen et al; BioTechniques, 34: 167-171 (2003). A large variety of such vectors are known in the art and are generally available.)

A “recombinant viral vector” refers to a viral vector comprising one or more heterologous genes or sequences. Since many viral vectors exhibit size-constraints associated with packaging, the heterologous genes or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying genes necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

Viral “packaging” as used herein refers to a series of intracellular events that results in the synthesis and assembly of a viral vector. Packaging typically involves the replication of the “pro-viral genome”, or a recombinant pro-vector typically referred to as a “vector plasmid” (which is a recombinant polynucleotide than can be packaged in an manner analogous to a viral genome, typically as a result of being flanked by appropriate viral “packaging sequences”), followed by encapsidation or other coating of the nucleic acid. Thus, when a suitable vector plasmid is introduced into a packaging cell line under appropriate conditions, it can be replicated and assembled into a viral particle. Viral “rep” and “cap” genes, found in many viral genomes, are genes encoding replication and encapsidation proteins, respectively. A “replication-defective” or “replication-incompetent” viral vector refers to a viral vector in which one or more functions necessary for replication and/or packaging are missing or altered, rendering the viral vector incapable of initiating viral replication following uptake by a host cell. To produce stocks of such replication-defective viral vectors, the virus or pro-viral nucleic acid can be introduced into a “packaging cell line” that has been modified to contain genes encoding the missing functions which can be supplied in trans). For example, such packaging genes can be stably integrated into a replicon of the packaging cell line or they can be introduced by transfection with a “packaging plasmid” or helper virus carrying genes encoding the missing functions.

A “detectable marker gene” is a gene that allows cells carrying the gene to be specifically detected (e.g., distinguished from cells which do not carry the marker gene). A large variety of such marker genes are known in the art. Preferred examples thereof include detectable marker genes which encode proteins appearing on cellular surfaces, thereby facilitating simplified and rapid detection and/or cellular sorting. By way of illustration, the lacZ gene encoding beta-galactosidase can be used as a detectable marker, allowing cells transduced with a vector carrying the lacZ gene to be detected by staining.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e. positive/negative) markers (see, e.g., WO 92/08796, published May 29, 1992, and WO 94/28143, published Dec. 8, 1994). Such marker genes can provide an added measure of control that can be advantageous in gene therapy contexts.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

The treatment of neoplastic disease or neoplastic cells, refers to an amount of the vectors and/or peptides, described throughout the specification and in the Examples which follow, capable of invoking one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion or (v) reducing, slowing or preventing metastasis; and/or (8) relief, to some extent, of one or more symptoms associated with the disorder.

Treatment of an individual suffering from an infectious disease organism refers to a decrease and elimination of the disease organism from an individual. For example, a decrease of viral particles as measured by plaque forming units or other automated diagnostic methods such as ELISA etc.

As used herein, a “pharmaceutically acceptable” component is one that is suitable for use with humans and/or animals without undue adverse side effects (such as toxicity, irritation, and allergic response) commensurate with a reasonable benefit/risk ratio.

“Cells of the immune system” or “immune cells” as used herein, is meant to include any cells of the immune system that may be assayed, including, but not limited to, B lymphocytes, also called B cells, T lymphocytes, also called T cells, natural killer (NK) cells, lymphokine-activated killer (LAK) cells, monocytes, macrophages, neutrophils, granulocytes, mast cells, platelets, Langerhans cells, stem cells, dendritic cells, peripheral blood mononuclear cells, tumor-infiltrating (TIL) cells, gene modified immune cells including hybridomas, drug modified immune cells, and derivatives, precursors or progenitors of the above cell types.

“Immune effector cells” refers to cells capable of binding an antigen and which mediate an immune response. These cells include, but are not limited to, T cells (T lymphocytes), B cells (B lymphocytes), monocytes, macrophages, natural killer (NK) cells and cytotoxic T lymphocytes (CTLs), for example CTL lines, CTL clones, and CTLs from tumor, inflammatory, or other infiltrates.

“Immune related molecules” refers to any molecule identified in any immune cell, whether in a resting (“non-stimulated”) or activated state, and includes any receptor, ligand, cell surface molecules, nucleic acid molecules, polypeptides, variants and fragments thereof.

“T cells” or “T lymphocytes” are a subset of lymphocytes originating in the thymus and having heterodimeric receptors associated with proteins of the CD3 complex (e.g., a rearranged T cell receptor, the heterodimeric protein on the T cell surfaces responsible for antigen/MHC specificity of the cells). T cell responses may be detected by assays for their effects on other cells (e.g., target cell killing, macrophage, activation, B-cell activation) or for the cytokines they produce.

“CD4” is a cell surface protein important for recognition by the T cell receptor of antigenic peptides bound to MHC class II molecules on the surface of an APC. Upon activation, naïve CD4 T cells differentiate into one of at least two cell types, Th1 cells and TH2 cells, each type being characterized by the cytokines it produces. “Th1 cells” are primarily involved in activating macrophages with respect to cellular immunity and the inflammatory response, whereas “Th2 cells” or “helper T cells” are primarily involved in stimulating B cells to produce antibodies (humoral immunity). CD4 is the receptor for the human immunodeficiency virus (HIV). Effector molecules for Th1 cells include, but are not limited to, IFN-γ, GM-CSF, TNF-α, CD40 ligand, Fas ligand, IL-3, TNF-β, and IL-2. Effector molecules for. Th2 cells include, but are not limited to, IL-4, IL-5, CD40 ligand, IL-3, GS-CSF, IL-10, TGF-β, and eotaxin. Activation of the Th1 type cytokine response can suppress the Th2 type cytokine response.

A “chemokine” is a small cytokine involved in the migration and activation of cells, including phagocytes and lymphocytes, and plays a role in inflammatory responses.

A “cytokine” is a protein made by a cell that affect the behavior of other cells through a “cytokine receptor” on the surface of the cells the cytokine effects. Cytokines manufactured by lymphocytes are sometimes termed “lymphokines.”

By the term “modulate,” it is meant that any of the mentioned activities, are, e.g., increased, enhanced, increased, agonized (acts as an agonist), promoted, decreased, reduced, suppressed blocked, or antagonized (acts as an agonist). Modulation can increase activity more than 1-fold, 2-fold, 3-fold, 5-fold, 10-fold, 100-fold, etc., over baseline values. Modulation can also decrease its activity below baseline values.

Other aspects of the invention are discussed infra.

### BRIEF DESCRIPTION OF THE DRAWINGS

FIG. 1 is a schematic illustration showing possible antigene lock conformations and target mechanism of antigene lock target binding. Antigene locks (blue) are shown in equilibrium between the native closed conformation (top left) and the open active conformation (top right). Note that the terminal bases of both arms are mispaired with the backbone. The gene target (red) may also be in equilibrium between double-stranded and denatured forms (middle). The antigene lock interacts with the denatured target (bottom right). Note the homology between the arms (including the terminal bases) and the target, while the mispairing between the lock and the target is in the backbone. The proposed structure of bound and ligated antigene lock is shown (bottom left). Since the backbone and combined arm lengths are 40 bases each, the antigene lock should be intertwined four times with each strand of the target DNA before being ligated.

FIG. 2A-2D show that the antigene lock structures bind specifically to their targets, and in the presence of DNA ligase, inhibit DNA synthesis in-vitro. FIG. 2A is a schematic of a plasmid showing the position of the pUC19 polylinker antigene lock (orange) on the target on the pUC19 plasmid (not drawn to scale). FIG. 2B, is a gel showing an Electrophoretic Mobility Shift Assay (EMSA) which demonstrates that the sequence specific antigene lock reacts with only the plasmid bearing the target sequence spontaneously at physiologic temperature. ³²P-labeled pUC19 antigene lock was incubated with pUC19 (lane 1), alone (lane 2), with pSG5 plasmid (lane 3) or pUC19-ΔPL (lane 4). FIG. 2C is a gel showing antigene lock binding was increased in the presence of DNA ligase. ³²P-labeled pUC19 antigene lock was incubated alone (lane 1), with pUC19 (lane 2) or with pUC19 and DNA ligase. FIG. 2D print out showing that DNA synthesis was arrested by the presence of the antigene lock during cycle sequencing. The antigene lock was mixed with pUC19, heated denatured, incubated with or without DNA ligase and cycle sequenced with either sequencing primer A or B.

FIG. 3A-3F are results showing production of white colonies after introduction of either lacZ or proA antigene locks have lost the F′ episome. FIG. 3A are photographs of colonies which were replica plated onto either Xgal plates containing (FIG. 3B) or lacking (FIG. 3C) exogenous proline. Note that the white colonies (e.g. numbers 3 and 4) were no longer capable of growth in the absence of exogenous proline (right). FIG. 3D is a gel showing PCR analysis of proA (lanes 2-5) and lacZ genes (lanes 6-10). Of five white colonies produced with either the lacZ or proA antigene locks, two representative colonies are shown. Note that both proA and lacZ gene PCR products were absent in episome preparations from the representative white, Pro⁻ colonies from lacZ antigene lock treated 8036/+6 cells (αlacZ 1 and 2). Lanes 1 and 6 contain 100 basepair DNA ladder, lanes 2 and 7 are no DNA PCR controls, and lanes 3 and 8 are positive PCR controls of purified wild-type episome. FIG. 3E is a graph showing that loss of β-galactosidase enzymatic activity correlated with Xgal staining results. β-galactosidase enzymatic activity measured in extracts of two representative white, Pro⁻ colonies generated with treatment of the proA (αPro1 and αPro2) or the lacZ (αlacZ1 and αlacZ2) antigene locks. Controls included WT (8036/wt episome) induced with IPTG, 8036 (αpro-lac), and 8036/+6 (Blue 1 and Blue 2). FIG. 3F shows the same two white Pro⁻ colonies generated by lacZ antigene lock treatment of 8036/+6 (αlacZ1 and 2) were conjugated with the wild-type episome, encoding an inducible phenotype distinct from the constitutive +6 phenotype. After conjugation, two white colonies were selected and streaked onto Xgal plates in the absence and presence of IPTG demonstrating the inducible phenotype. The ability of the cells to exhibit the novel phenotype is consistent with their having been female and taken up the novel wild-type episome. Control colonies were from 8036/+6.

FIG. 4A-4F shows that both lacZ and proA antigene locks selectively kill cells containing their targets in the host chromosome. FIG. 4A-4E are photographs showing selective killing of HB101 (pro⁺, lacZ⁺) cells with the lacZ and proA antigene locks. Mixtures of HB101 (containing the wild-type lac operon in the chromosome) and 8036 (with a deletion of lacZ and proA genes) were mixed and plated on Xgal with ampicillin and IPTG (FIG. 4A, control untreated). When exposed to either the lacZ or proA antigene locks in the presence of IPTG, a selective loss of β-galactosidase positive colonies was seen (FIGS. 4D and 4E). This was not observed with either of the control randomized antigene locks (FIGS. 4B and 4C). FIG. 4F is a bar chart comparing the specific cell kill of HB101 in the HB101/8036 cell mix using the sequence specific lacZ and proA antigene locks and their controls. Percent colony reduction of HB101 cells after transformation with specific or control antigene locks was calculated as follows: 1−(observed blue colonies)/# expected, where # expected=(# white colonies, no lock)/((# whites colonies, lock treated)*(# blue colonies, no lock)). The data represents the means of 5 independent experiments and error bars, standard error of the mean. p values were calculated using a paired t-test.

FIG. 5 are photographs showing that the antigene locks are active irrespective of the transcriptional status of the gene. The HB101/8036 cell mixture was exposed to the lacZ antigene lock (bottom panel) or lacZ control (top panel) lock in the absence of IPTG (left plates of each panel), and then replica-plated onto Xgal plates with IPTG (right plates of each panel). Note that the relative reduction in blue colonies is still seen in the bottom panel, despite that the cells were lacZ antigene lock treated when the gene was repressed.

FIGS. 6A-6C show that gene specific antigene locks can kill human cervical cancer cells. FIG. 6A is a schematic illustration showing the position of the antigene locks (red) on their targets on the alu repeat and the HPV-16 E7 oncogene (not drawn to scale). FIG. 6B is a bar graph showing gene specific alu antigene lock specifically kills human cervical cancer cells. Reduction in colony count was monitored after transfection with the alu sequence specific or control alu antigene locks in three human cervical cancer or A9 mouse cell lines. Bars represent the means of 3 independent experiments and error bars, standard error of the mean. p values were calculated using a paired t-test. FIG. 6C is a bar graph showing gene specific E7 antigene lock selectively kills human cervical cancer cells, CaSki and C33A/E7, which contain the E7 gene target. Reduction of in colony count after transfection with the E7 sequence-specific or control E7 antigene locks was determined in the three cervical cancer cell lines.

FIG. 7A-7F shows the production of white colonies when 8036/+6 cells are transformed with either lacZ or proA anti-gene locks. FIG. 7A is a schematic illustration showing the position of the anti-gene locks (orange) on their targets on the lacZ and proA genes (not drawn to scale). FIGS. 7B-7E shows the production of white colonies (arrows) when 8036/+6 cells were transformed with either lacZ or proA anti-gene locks. Phosphorylated lacZ or proA anti-gene locks (FIGS. 7C and 7E) or lacZ or proA control anti-gene locks (FIGS. 7B and 7D) were co-transformed with pSG5 plasmid, at a molar ratio of 9000:1 (anti-gene lock: plasmid), into competent E. coli 8036/+6 cells, and plated out on Xgal plates containing exogenous proline. Note the absence of sectored colonies. FIG. 7F is a bar graph comparing the production of white colonies in 8036/+6 after transformation with the sequence specific lacZ and proA anti-gene locks and their controls. The data represents the means of 4 independent experiments and error bars, standard error of the mean. p values were calculated using a paired t-test.

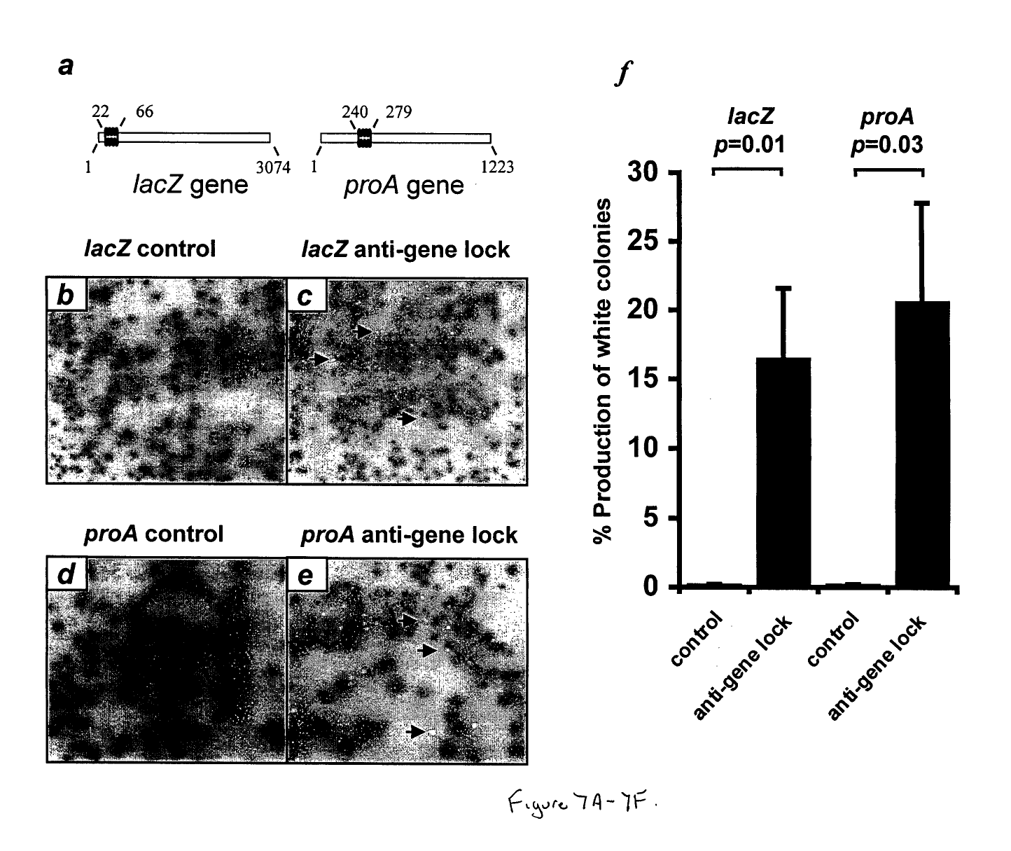

### DETAILED DESCRIPTION OF THE INVENTION

The invention provides methods for selective killing of cells based on their genotype. Such methods utilize molecules termed, herein, “Antigene Locks.” Antigene Locks bind specifically to their gene targets, intertwine with both strands of the target DNA and are irreversibly ligated (“locked”), thereby inhibiting DNA synthesis. When transformed into a mixed population of cells, where only one cell type possesses the target, antigene locks selectively kill only the target bearing cell population. Antigene locks kill cells irrespective of their transcriptional status, and are active in both prokaryotic and eukaryotic cells. Another preferred use is manipulation of cell strains causing plasmids and episomes to be eliminated from cells.

In a first aspect, the invention provides methods for treating cells comprising an infectious agent. Such treatment methods comprise administering an antigene oligonucleotide to cells that comprise an oligonucleotide sequence of an infectious agent. The antigene oligonucleotide preferably will be complementary to the infectious agent oligonucleotide sequence. A variety of cells may be treated in accordance with such methods, and typically mammalian cells are treated, especially primate cells such as human cells.

In accordance with the invention target cells, either prokaryotic and eukaryotic, are selectively targeted by an antigene lock based on their genetic makeup. Infectious disease almost invariably results in the acquisition of foreign nucleic acids, which could be targeted using this technology. Specific targets could be viral, e.g. HIV (virus or provirus) or bacterial, e.g. multi-drug resistant bacteria e.g. TB, fungal or protoazoan. This technology can be especially useful in treating infections for which there is no effective anti-microbial or anti-viral agent (e.g. Ebola virus, etc.), or known or novel bio-terrorist agents.

Preferred antigene locks of the invention will hybridize (bind) to a target sequence, particularly a target oligonucleotide of an infectious agent such as a viral, bacterial, fungal or protozoan agent including those agents and sequences disclosed herein, under stringency conditions as may be assessed in vitro. Such conditions are disclosed and defined below.

The invention may be used against protein coding genes as well as non-protein coding genes. Examples of non-protein coding genes include genes that encode ribosomal RNAs, transfer RNAs, small nuclear RNAs, small cytoplasmic RNAs, telomerase RNA, RNA molecules involved in DNA replication, chromosomal rearrangement and the like.

In another preferred embodiment, abnormal or cancer cells are targeted by the antigenes. For example, many malignancies are associated with the presence of foreign DNA, e.g. Bcr-Abl, Bcl-2, HPV, and these provide unique molecular targets to permit selective malignant cell targeting. The approach can be used to target single base substitutions (e.g. K-ras, p53) or methylation changes. However, proliferation of cancer cells may also be caused by previously unexpressed genes. These gene sequences can be targeted, thereby, inhibiting further expression and ultimate death of the cancer cell. In other instances, transposons can be the cause of such deregulation and transposon sequences can be targeted, e.g. Tn5.

According to the present invention, an antigene oligonucleotide is designed to be specific for a gene, which either causes, participates in, or aggravates a disease state, in a patient. For example, viral infection, and an antigene lock can be targeted against to genes responsible for viral replication; a viral infection cycle, such as, for example, attachment to cellular ligands; viral genes encoding host immune modulating functions. Particularly preferred viral organisms causing human diseases according to the present invention include (but not restricted to) Filoviruses, Herpes viruses, Hepatitisviruses, Retroviruses, Orthomyxoviruses, Paramyxoviruses, Togaviruses, Picornaviruses, Papovaviruses and Gastroenteritisviruses. Other preferred, non-limiting examples of viral agents are listed in Table 1.

According to another preferred embodiment of the invention, the antigene oligonucleotide is specific for human or domestic animal bacterial pathogens. Particularly preferred bacteria causing serious human diseases are the Gram positive organisms: Staphylococcus aureus, Staphylococcus epidermidis, Enterococcus faecalis and E. faecium, Streptococcus pneumoniae and the Gram negative organisms: Pseudomonas aeruginosa, Burkholdia cepacia, Xanthomonas maltophila, Escherichia coli, Enterobacter spp, Klebsiella pneumoniae and Salmonella spp. The target genes may include (but are not restricted to) genes essential to bacterial survival and multiplication in the host organism, virulence genes, genes encoding single- or multi-drug resistance. However, gram negative bacteria are also within the scope of the invention.

In another preferred embodiment, the antigene locks are targeted to toxins produced by a disease agent such as anthrax. For example, anthrax which is one of the agents that can be used in a bioterrorist attack. Anthrax infection is mediated by spores of Bacillus anthracis, which can gain entry to the body through breaks in the skin, through inhalation, or through ingestion. Fatal anthrax is characterized by the establishment of a systemic bacteremia that is accompanied by an overwhelming toxemia. It seems that anthrax is a 2-pronged attack with the bacteremia and/or toxemia contributing to the fatal syndrome of shock, hypoperfusion, and multiple organ system failure. The likelihood of developing systemic disease varies with the portal of organism entry, and is most pronounced for the inhalational route (reviewed in Dixon et al., 1999, New England J. Med. 341: 815-826). Antigene oligonucleotides can be targeted to the genes that inhibit proliferation of the bacteria in an infected patient and target the toxin producing genes thereby eliminating the toxic effects of the anthrax infection. Alternatively, antigene locks could be targeted to any sequence target that is present in the organism and lacking in the host.

According to one preferred embodiment of the invention, the antigene oligonucleotide is specific for protozoa infecting humans and causing human diseases. Particularly preferred protozoan organisms causing human diseases according to the present invention include (but not restricted to) Malaria e.g. Plasmodium falciparum and M. ovale, Trypanosomiasis (sleeping sickness) e.g. Trypanosoma cruzei, Leischmaniasis e.g. Leischmania donovani, Amebiasis e.g. Entamoeba histolytica.

According to one preferred embodiment of the invention, the antigene oligonucleotide is specific for fungi causing pathogenic infections in humans. Particularly preferred fungi causing or associated with human diseases according to the present invention include (but not restricted to) Candida albicans, Histoplasma neoformans, Coccidioides immitis and Penicillium marneffei.

The invention in general provides a method for treating diseases, such as cancer and diseases which are caused by infectious agents such as viruses, bacteria, intra- and extra-cellular parasites, insertion elements, fungal infections, etc., which may also cause expression of genes by a normally unexpressed gene, abnormal expression of a normally expressed gene or expression of an abnormal gene, comprising administering to a patient in need of such treatment an effective amount of an antigene oligonucleotide; or a cocktail of different modified antigene locks; or a cocktail of different modified and unmodified antigene oligonucleotides specific for the disease causing entity.

In accordance with the invention, antigene oligonucleotide therapies comprise administered antigene oligonucleotide which contacts (interacts with) the targeted gene or mRNA from the gene, whereby expression of the gene is modulated, and expression is inhibited. Such modulation of expression suitably can be a difference of at least about 10% or 20% relative to a control, more preferably at least about 30%, 40%, 50%, 60%, 70%, 80%, or 90% difference in expression relative to a control. It will be particularly preferred where interaction or contact with an antigene oligonucleotide results in complete or essentially complete modulation of expression relative to a control, e.g., at least about a 95%, 97%, 98%, 99% or 100% inhibition of or increase in expression relative to control. A control sample for determination of such modulation can be comparable cells (in vitro or in vivo) that have not been contacted with the antigene oligonucleotide.

The methods of the invention are preferably employed for treatment or prophylaxis against diseases caused abnormal cell growth and by infectious agents, particularly for treatment of infections as may occur in tissue such as lung, heart, liver, prostate, brain, testes, stomach, intestine, bowel, spinal cord, sinuses, urinary tract or ovaries of a subject. The methods of the invention also may be employed to treat systemic conditions such as viremia or septicemia. The methods of the invention are also preferably employed for treatment of diseases and disorders associated with viral infections or bacterial infections, as well as any other disorder caused by an infectious agent.

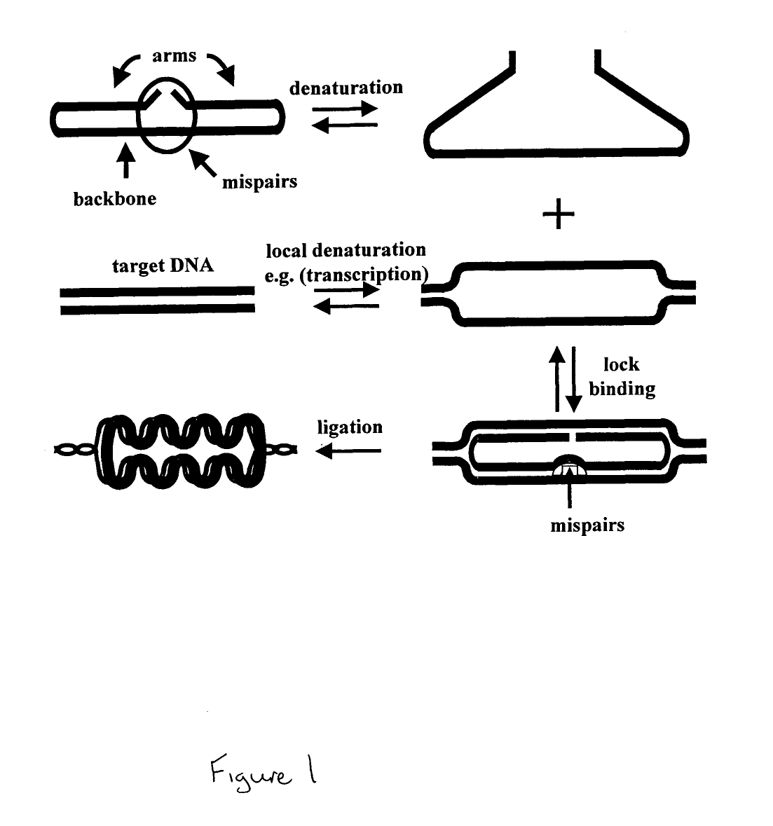

Preferably, a disease agent is isolated from a patient and identified using diagnostic tools such as ELISA's RIAs, cell sorting, PCR and the like. However, a disease causing agent may be a novel agent to which antigene oligonucleotides can be targeted. Sequencing data obtained from the agent can be used to construct an antigene lock. Partial sequencing of the agent can be accomplished by any means known in the art. As an illustrative example which is not meant to limit or construe the invention in any way, the following is provided. The antigene lock is designed to be complementary to selected sequences. For example, the backbone and the arms are constructed so that they are complementary to both target DNA strands, and therefore to each other (FIG. 1a, top left). Without wishing to be bound by theory, in the cell an equilibrium exists between this closed inactive form and an active open form in which the antigene lock is denatured (FIG. 1a, top right). In the denatured form, the backbone and arms would have the ability to bind to both strands of locally denatured target DNA, creating two DNA duplexes, using Watson and Crick pairing (FIG. 1a, bottom right). If such structures are ligated in the cell, both target DNA strands should be inextricably intertwined with the lock (FIG. 1a, bottom left). Such structures (or complexes) would be unable to denature as is required during either transcription or replication. These complexes would likely be more resistant to single stranded cellular exonucleases, increasing the probability of reaching their DNA targets.

To inhibit DNA ligase activity from acting on these structures in the unbound state, since both arms bound to the backbone with the terminal bases are juxtaposed, and inactivating the antigene lock, mispairs are created between the terminal bases of the arms and the backbone (FIG. 1a, top left). This is accomplished by making the base changes in the backbone rather than in the arms so that the arms maintain full complementarity to the target gene (FIG. 1a, bottom right).

After reacting with their DNA or RNA target, antigene locks may be ligated by, for example, native cellular ligases. Alternatively, the ends of the antigene locks may be chemically modified such that they self-ligate when the ends are juxtaposed on their specific target. See, for example, Sando and Kool, J. Am. Chem. Soc., 124: 9686-9687, 2002 which is incorporated herein, in its entirety. Examples of chemical modifications include, but are not limited to: dabsyl and thioate moeities.

In another preferred embodiment, the antigene locks comprise molecules or oligonucleotide sequences comprising ligase activity. For example, PCR products are cloned, using standard TA cloning, but in which a vector is designed to comprise topoisomerase recognition sequences (e.g. CCCTT), and in which topoisomerases (e.g. topoisomerase I isolated from Vaccinia), comprising ligase activity is covalently ligated to the cloning vector (Shuman et al, J. Biol. Chem., 269: 32678-32684, 1994; Heyman et al, Genome Research, 9: 383-392, 1999). Similarly, a ligase or topoisomerase or other enzyme possessing ligase activity could be covalently attached to the antigene locks to facilitate ligation after target binding.

The antigene locks, disclosed herein, are different from other molecular approaches taken in the prior art:

### Anti-RNA Approaches:

Triple helix forming oligonucleotides (TFO or triplexes) are single stranded DNA oligonucleotides, approximately 15-25 bases in length, which bind to a specific region of the gene causing the “triple helix” effect. They need to target homopurine/homopyrimidine tracts exclusively and the 5′ and 3′ ends do not undergo ligation. Antigene locks are longer than 25 bases and have the ability to become ligated. TFO's only bind one strand of target DNA whereas antigene locks are circularizing locking oligonucleotides. Antigene locks do not have a sequence restriction for binding. TFO's do not induce cell death, they inhibit gene expression, whereas the antigenes disclosed herewith, induce cell death.

### Antisense RNA and DNA:

Antisense nucleic acids are typically single stranded oligonucleotides which are complementary to mRNA and block mRNA expression by either inhibiting nuclear to cytoplasmic transport, ribosome binding or translation. Antigene locks are different from the antisense approach because they target the actual gene. Antisense RNA and DNA target the mRNA. With antigene locks, if the cell is not killed, mRNA production will be inhibited whereas cells are not killed using the antisense approach. The structure of the antisense RNA and DNA (linear oligonucleotides) are not the same as the antigene locks (circularizing oligonucleotides).

### Ribozymes:

Ribozymes, linear oligonucleotides with a loop structure, are catalytic nucleic acids, which are designed to inactivate specific mRNA. Antigene locks do not possess catalytic properties and are very different in structure. Ribozymes are dependent on magnesium, antigene locks are not.

### DNAzymes:

DNAzymes are catalytic nucleic acids that bind to and cleave RNA. Antigene locks are not catalytic and do not cleave RNA. They are very different in structure. DNAzymes are dependent on magnesium, antigene locks are not.

### Circular Oligonucleotides:

The structure of antigene locks resembles but is very different from the structure of circular oligonucleotides (CO). Circular oligonucleotides possess two non-complementary strands of DNA joined by two hinges containing 5 thymidine bases each. They work by surrounding and binding to one strand of DNA. Antigene locks are proposed to bind both strands of DNA but most importantly are not circular but have the ability to circularize and undergo ligation. Circular oligonucleotides do not. There is mispairing between the terminal bases and the backbone in the antigene locks. Antigene locks are anticipated to intertwine both strands of the target DNA approximately four times before ligation. Circular oligonucleotides just bind one strand.

### Padlock Probes:

Antigenes are structurally and functionally different from padlock probes in critical ways:

Padlock probes are designed as an in vitro tool for in situ hybridization and not as therapeutics.

The backbone and the arms of the disclosed antigene locks are complementary to each other and complementary to both strands of the double stranded DNA target. Therefore, the antigene locks bind and inter twine with both strands of the double stranded DNA target. Padlock probes only bind to one DNA strand of their target and the backbone is not complementary to the arms.

The terminal bases of the arms of the disclosed antigenes are mispaired with the corresponding bases in the backbone so that self-ligation of the antigene locks does not occur. The whole sequence of the backbone is mispaired with the arms of the padlock probes.

According to one preferred embodiment of the invention, the nucleobases in the antigene lock may be modified to provided higher specificity and affinity for a target gene. For example nucleobases may be substituted with LNA monomers, which can be in contiguous stretches or in different positions. The modified antigene, preferably has a higher association constant (Kₐ) for the target sequences than the complementary sequence. Binding of the modified or non-modified antigene locks to target sequences can be determined in vitro under a variety of stringency conditions using hybridization assays and as described in the examples which follow.

A fundamental property of oligonucleotides that underlies many of their potential therapeutic applications is their ability to recognize and hybridize specifically to complementary single stranded nucleic acids employing either Watson-Crick hydrogen bonding (A-T and G-C) or other hydrogen bonding schemes such as the Hoogsteen/reverse Hoogsteen mode. Affinity and specificity are properties commonly employed to characterize hybridization characteristics of a particular oligonucleotide. Affinity is a measure of the binding strength of the oligonucleotide to its complementary target (expressed as the thermostability (Tₘ) of the duplex). Each nucleobase pair in the duplex adds to the thermostability and thus affinity increases with increasing size (No. of nucleobases) of the oligonucleotide. Specificity is a measure of the ability of the oligonucleotide to discriminate between a fully complementary and a mismatched target sequence. In other words, specificity is a measure of the loss of affinity associated with mismatched nucleobase pairs in the target.

The utility of an antigene oligonucleotide for modulation (including inhibition) of expression of a targeted gene can be readily determined by simple testing. Thus, an in vitro or in vivo expression system comprising the targeted gene, mutations or fragments thereof, can be contacted with a particular antigene oligonucleotide (modified or un modified) and levels of expression are compared to a control, that is, using the identical expression system which was not contacted with the antigene oligonucleotide.

Antigene oligonucleotides may be used in combinations. For instance, a cocktail of several different antigene modified and/or unmodified oligonucleotides, directed against different regions of the same gene, may be administered simultaneously or separately.

According to one preferred embodiment, the antigene is specific for genes responsible for viral replication; viral infection cycle such as attachment to cellular ligands; viral genes encoding host immune modulating functions. Examples of viral organisms include, but not restricted to, those listed in table 1. For information about the viral organisms see Fields of Virology, 3. ed., vol 1 and 2, BN Fields et al. (eds.). Non-limiting examples of targets of selected viral organisms are listed in table 2.

TABLE 1
Selected viral organisms causing human diseases.

| | |
|---|---|
| Herpesviruses | |
| | Alpha-herpesviruses: |
| | Herpes simplex virus 1 (HSV-1) |
| | Herpes simplex virus 2 (HSV-2) |
| | Varicella Zoster virus (VZV) |
| | Beta-herpesviruses: |
| | Cytomegalovirus (CMV) |
| | Herpes virus 6 (HHV-6) |
| | Gamma-herpesviruses: |
| | Epstein-Barr virus (EBV) |
| | Herpes virus 8 (HHV-8) |
| Hepatitis viruses | |
| | Hepatitis A virus |
| | Hepatitis B virus |
| | Hepatitis C virus (see Example 4) |
| | Hepatitis D virus |
| | Hepatitis E virus |
| Retroviruses | |
| | Human Immunodeficiency 1 |
| | (HIV-1)(see Example 3) |
| Orthomyxoviruses | |
| | Influenzaviruses A, B and C |
| Paramyxoviruses | |
| | Respiratory Syncytial virus (RSV) |
| | Parainfluenza viruses (PI) |
| | Mumps virus |
| | Measles virus |
| Togaviruses | |
| | Rubella virus |
| Picornaviruses | |
| | Enteroviruses |
| | Rhinoviruses |
| | Coronaviruses |
| Papovaviruses | |
| | Human papilloma viruses (HPV) |
| | Polyomaviruses (BKV and JCV) |
| Gastroenteritisviruses | |
| Filoviridae | |
| Bunyaviridae | |
| Rhabdoviridae | |
| Flaviviridae | |

TABLE 2
Target genes of viral organisms

| Organism | target gene | open reading frame | gene product |
|---|---|---|---|
| HIV | gag: | MA | p17 |
| | | CA | p24 |
| | | NC | p7 |
| | | | p6 |
| | pol: | PR | p15 |
| | | RT | p66 |
| | | | p31 |
| | env: | | gp120 |
| | | | gp41 |
| | tat | | transcriptional transactivator |
| | rev | | regulator of viral expression |
| | vif | | |
| | vpr | | |
| | vpu | | |
| | nef | | |
| RSV | NS1 | | |
| | NS2 | | |
| | L | | |
| | 2-5A-dependent Rnase L | | |
| HPV | E1 | | helicase |
| | E2 | | transcription regulator |
| | E3 | | |
| | E4 | | late NS protein |
| | E5 | | transforming protein |
| | E6 | | transforming protein |
| | E7 | | transforming protein |
| | E8 | | |
| | L1 | | major capsid protein |
| | L2 | | minor capsid protein |
| HCV | NS3 | | protease |
| | NS3 | | helicase |
| | HCV-IRES | | (see Example 4) |
| | NS5B | | polymerase |
| HCMV | DNA polymerase | | |
| | IE1 | | |
| | IE2 | | |
| | UL36 | | |
| | UL37 | | |
| | UL44 | | polymerase asc. protein |
| | UL54 | | polymerase |
| | UL57 | | DNA binding protein |
| | UL70 | | primase |
| | UL102 | | primase asc. protein |
| | UL112 | | |
| | UL113 | | |
| | IRS1 | | |
| VZV | | 6 | |
| | | 16 | |
| | | 18 | |
| | | 19 | |
| | | 28 | |
| | | 29 | |
| | | 31 | |
| | | 39 | |
| | | 42 | |
| | | 45 | |
| | | 47 | |
| | | 51 | |
| | | 52 | |
| | | 55 | |
| | | 62 | |
| | | 71 | |
| HSV | IE4 | | US1 |
| | IE5 | | US12 |
| | IE110 | | ICP0 |
| | IE175 | | ICP4 |
| | UL5 | | helicase |
| | UL8 | | helicase |
| | UL13 | | capsid protein |
| | UL30 | | polymerase |
| | UL39 | | ICP6 |
| | UL42 | | DNA binding protein |

Information about the above selected genes, open reading frames and gene products is found in the following references: Field A. K. and Biron, K. K. “The end of innocence” revisited: resistance of herpesviruses to antiviral drugs. Clin. Microbiol. Rev. 1994; 7: 1-13. Anonymous. Drug resistance in cytomegalovirus: current knowledge and implications for patient management. J. Acquir. Immune Defic. Syndr. Hum. Retrovir. 1996; 12: S1-SS22. Kelley R et al. Varicella in children with perinatally acquired human immunodeficiency virus infection. J Pediatr 1994; 124: 271-273. Hanecak et al. Antisense oligonucleotides inhibition of hepatitis C virus. gene expression in transformed hepatocytes. J Virol 1996; 70: 5203-12. Walker Drug discovery Today 1999; 4: 518-529. Zhang et al. Antisense oligonucleotides inhibition of hepatitis C virus (HCV) gene expression in livers of mice infected with an HCV-Vaccinia virus recombinant. Antim. Agents Chemotherapy 1999; 43, 347-53. Feigin R D, Chemy J D, eds. Textbook of pediatric infectious diseases. Philadelphia: WB Saunders, 1981. Chen B. et al., Induction of apoptosis of human cervical carcinoma cell line SiHa by antisense oligonucleotide og human papillomavirus type 16 E6 gene. 2000; 21(3): 335-339. The human herpesviruses. New York: Raven Press; 1993. DeClerque E, Walker R T, eds. Antiviral drug development: a multi-disciplinary approach. Plenum; 1987. Antiviral Drug Resistance (Richman, D. D., ed.), Wiley, Chichester, 1995. Flint S J et al. eds. Principles of virology: Molecular biology, pathogenesis and control.

It should be appreciated that in the above table 2, an indicated gene means the gene and all currently known variants thereof, including the different mRNA transcripts that the gene and its variants can give rise to, and any further gene variants which may be elucidated. In general, however, such variants will have significant sequence identity to a sequence of table 2 above, e.g. a variant will have at least about 70 percent sequence identity to a sequence of the above table 2, more typically at least about 75, 80, 85, 90, 95, 97, 98 or 99 percent sequence identity to a sequence of the above table 2. Sequence identity of a variant can be determined by any of a number of standard techniques such as a BLAST program http://www.ncbi.nlm.nih.gov/blast/).

Sequences for the genes listed in Table 2 can be found in GenBank (http://www.ncbi.nlm.nih.gov/). The gene sequences may be genomic, cDNA or mRNA sequences. Preferred sequences are viral genes containing the complete coding region and 5′ untranslated sequences that are involved in viral replication.

In vitro propagation of virus causing human diseases: To screen for antiviral effect of antigene oligonucleotides viral particles are propagated in in vitro culture systems of appropriate mammalian cells. Initial screening is typically performed in transformed cell lines. More thorough screening is typically performed in human diploid cells. Detailed methods of screening are described in the Examples which follow.

TABLE 3
Examples of in vitro propagation of viruses.

| Organism | WI-38 or MRC-5 | HeLa or HEp-2 | PRMK or PCMK |
|---|---|---|---|
| HSV | C, D, S | D | D |
| HCMV | C, F | — | — |
| VZV | C, F | — | — |
| Adeno | D | D | D |
| RSV | S | S | S |
| Polio | D | D | D |
| Echo | D | — | D |
| Rhino | D, F | — | D, F |

C is cytomegaly, D is cell destruction, F is marked focality, H is hemadsorption and S is formation of syncytium. “—” means that the cell line does not sustain growth of the virus. WI-38 is a human diploid fibroblast cell line. MRC-5 is human lung fibroblasts. HeLa is a human aneuploid epithelial cell line. PRMK is primary rhesus monkey kidney cells. PCMK is primary cynomolgus monkey kidney cells.

Likewise Vero cells (green monkey kidney cells) and Mewo cells will sustain the growth of for example herpesviruses. References: DeClerque E, Walker R T, eds. Antiviral drug development: a multi-disciplinary approach. Plenum; 1987. Antiviral Drug Resistance (Richman, D. D., ed.), Wiley, Chichester, 1995. Cytomegalovirus protocols, J. Sinclair (ed.), Humana Press. HIV Protocols, N. Michael and J H Kim (eds.), Humana press. Hepatitis C Protocols, JYN Lau (ed.), Humana Press. Antiviral Methods and Protocols, D Kinchington and RF Schinazi, Humana Press.

Bacterial infections: According to another preferred embodiment of the invention, the antigene oligonucleotide is specific for the human or domestic animal bacterial pathogens listed in (but not restricted to) table 4. The target genes may include (but are not restricted to) genes essential to bacterial survival and multiplication in the host organism, virulence genes, genes encoding single- or multi-drug resistance such as for instance the genes listed in table 5.

TABLE 4
Selected bacteria causing serious human diseases

| | |
|---|---|
| Gram positive organisms: | |
| | Staphylococcus aureus: strains include |
| | methicillin resistant (MRSA), methicillin- |
| | vancomycin resistant (VMRSA) and |
| | vancomycin intermediate resistant (VISA). |
| | Staphylococcus epidermidis. |
| | Enterococcus faecalis and E. faecium: strains |
| | include vancomycin resistant (VRE). |
| | Streptococcus pneumoniae. |
| Gram negative organisms: | |
| | Pseudomonas aeruginosa. |
| | Burkholdia cepacia. |
| | Xanthomonas maltophila. |
| | Escherichia coli |
| | Enterobacter spp. |
| | Klebsiella pneumoniae |
| | Salmonella spp. |

References: Cookson B. D., Nosocomial antimicrobial resistance surveillance. J. Hosp. Infect. 1999:97-103. Richards M. J. et al. Nosocomial infections in medical intensive care units in the United States. National Nosocomial Infections Surveillance System. Crit. Care. Med. 1999; 5:887-92. House of Lords Select Committee on Science and Technology. Resistance to antibiotics and other antimicrobial agents. London: 1998; Her Majesty's Stationary Office. Johnson A. P. Intermediate vancomycin resistance in S. aureus: a major threat or a minor inconveniance? J. Antimicrobial. Chemother. 1998; 42:289-91. Baquero F. Pneumococcal resistance to beta-lactam antibiotics: a global overview. Microb. Drug Resist. 1995; 1:115-20. Hsuch P. R. et al. Persistence of a multidrug resistant Pseudomonas aeruginosa clone in an intensive care burn unit. J. Clin. Microbiol. 1998; 36:1347-51. Livermore D. Multiresistance and Superbugs. Commun. Dis. Public Health 1998; 1:74-76.

The preferred target genes in bacteria would include (but are not restricted to) genes involved in the following biological functions: 1. Protein synthesis; 2. Cell wall synthesis; 3: Cell division; 4: Nucleic acid synthesis; and 5: Virulence. The biological functions mentioned are analogous in Gram positive and Gram negative bacteria, and the genes encoding the individual proteins involved may exhibit extensive homologies in their nucleotide sequences. The genes encoding the mentioned target complexes may have different names in different bacteria.

TABLE 5
Examples of selected antigene target complexes in bacteria.

| | |
|---|---|
| Protein synthesis targets | Translation initiation factors (e.g. IF1, IF2, IF3) |
| | Translation elongation factors (e.g. EF-Tu, EF-G) |
| | Translation release factors (RF1, RF2, RF3) |
| Cell wall synthesis | Penicillin binding proteins (e.g. PBP1 to PBP9) |
| Cell division | Proteins encoded by the ftsQAZ operon |
| Nucleic acid synthesis | Gyrases, Sigma 70 and Helicase |
| Virulence | Ureases |

References: Escherichia coli and Salmonella in Cellular and Molecular Biology, vol 1 & 2. C Neidhardt and R Curtiss (eds.), American Society for Microbiology Press. Gram-Positive Pathogens. V A Fischetti et al. (eds.), American Society for Microbiology Press. Bacterial Pathogenesis: A Molecular Approach. A A Salyers and D D Whitt (eds.), American Society for Microbiology Press. Organization of the Procaryotic Genome. RL Charlebois (ed.), American Society for Microbiology Press.

Listed in Table 6 below are examples of genes encoding the protein complexes listed in Table 5 above. The individual genes have homologues in the major human pathogenic bacteria listed in Table 4. Table 6 below depicts an example of a Gram negative (Escherichia coli) and a Gram positive (Staphylococcus aureus) organism, chosen as representatives for the two groups of bacteria.

TABLE 6
Examples of genes encoding possible antigene target proteins.

| Target group | E. coli | S. aureus |
|---|---|---|
| Protein synthesis | prfA | prfA |
| | prfB | |
| | prfC | prfC |
| | infA | infA |
| | infB | infB |
| | infC | |
| | tufA | tuf |
| | fusA | fus |
| Cell wall synthesis | mrcA | pbpA |
| | mrcB | pbp2 |
| | pbpB | fmhB |
| | | femA |
| | | femB |
| Cell division | ftsA | ftsA |
| | ftsQ | |
| | ftsZ | ftsZ |
| Nucleic acid synthesis | gyrA | perC |
| | gyrB | |
| | rpoD | |

References: Escherichia coli and Salmonella in Cellular and Molecular Biology, vol 1 & 2. C Neidhardt and R Curtiss (eds.), American Society for Microbiology Press. Gram-Positive Pathogens. V A Fischetti et al. (eds.), American Society for Microbiology Press. Bacterial Pathogenesis: A Molecular Approach. A A Salyers and D D Whitt (eds.), American Society for Microbiology Press. Organization of the Procaryotic Genome. RL Charlebois (ed.), American Society for Microbiology Press.

Related bacterial species among the Gram negatives as well as the Gram positives exhibit homologous genes that serve as antigene targets.

It should be appreciated that in the above table 5 and 6, an indicated gene means the gene and all currently known variants thereof, including the different mRNA transcripts that the gene and its variants can give rise to, and any further gene variants which may be elucidated. In general, however, such variants will have significant sequence identity to a sequence of table 5 and 6 above, e.g. a variant will have at least about 70 percent sequence identity to a sequence of the above table 5 and 6, more typically at least about 75, 80, 85, 90, 95, 97, 98 or 99 percent sequence identity to a sequence of the above table 5 and 6. Sequence identity of a variant can be determined by any of a number of standard techniques such as a BLAST program http://www.ncbi.nlm.nih.gov/blast/).

Sequences for the genes listed in Table 5 and 6 can be found in GenBank (http://www.ncbi.nlm.nih.gov/). The gene sequences may be genomic, cDNA or mRNA sequences. Preferred sequences are viral genes containing the complete coding region and 5′ untranslated sequences that are involved in viral replication.

Protozoan infections: According to one preferred embodiment of the invention, the antigene oligonucleotide is specific for protozoan organisms infecting humans and causing human diseases. Such protozoa include, but are not restricted to, the following: 1. Malaria e.g. Plasmodium falciparum and M. ovale. (references: Malaria by M Wahlgren and P Perlman (eds.), Harwood Academic Publishers, 1999. Molecular Immunological Considerations in Malaria Vaccine Development by M F Good and A J Saul, CRC Press 1993). 2. Trypanosomiasis (sleeping sickness) e.g. Trypanosoma cruzei (reference: Progress in Human African Trypanosomiasis, Sleeping Sickness by M Dumas et al. (eds.), Springer Verlag 1998). 3. Leischmaniasis e.g. Leischmania donovani (reference: AL Banuals et al., Molecular Epidemiology and Evolutionary Genetics of Leischmania Parasites. Int J Parasitol 1999; 29:1137-47). 4. Amebiasis e.g. Entamoeba histolytica (RP Stock et al., Inhibition of Gene Expression in Entamoeba histolytica with Antisense Peptide Nucleic Acid Oligomers. Nature Biotechnology 2001; 19:231-34).

Fungal infections: According to one preferred embodiment of the invention, the antigene oligonucleotide is specific for fungi cause pathogenic infections in humans. These include, but are not restricted to, the following: Candida albicans (references: AH Groll et al., Clinical pharmacology of systemic antifungal agents: a comprehensive review of agents in clinical use, current investigational compounds, and putative targets for antifungal drug development. Adv. Pharmacol. 1998:44:343-501. MDD Backer et al., An antisense-based functional genomics approach for identification of genes critical for growth of Candida albicans. Nature Biotechnology 2001; 19:235-241) and others, e.g., Histoplasma neoformans, Coccidioides immitis and Penicillium marneffei (reference: SA Marques et al., Mycoses associated with AIDS in the Third World. Med. Mycol 2000; 38 Suppl 1:269-79).

Host cellular genes involved in viral diseases: According to one preferred embodiment of the invention, the antigene oligonucleotide is specific for host cellular genes involved in viral diseases. Besides genes encoded by viruses for their replication, the initial step to infection is binding to cellular ligands. For example CD4, chemokine receptors such as CCR3, CCR5 are required for HIV infection. Furthermore, viruses also upregulate certain chemokines which aid in their replication, for example in the case of HIV there is an increase in IL-2 which results in an increase of CD4⁺ T cells, allowing for an increase in the pool of cells for further infection in the early stages of the disease. The antigene oligonucleotides may be used to prevent any further upregulation of genes that may aid in the infectivity and replication rate of the viruses. Preferred targets are the 5′ untranslated sequences of ligands used by viruses to infect a cell, or any other cellular factor that aids in the replication of the viruses. Particularly preferred are human cDNA sequences. According to the invention antigene oligonucleotides may be used to modulate the expression of genes involved in the viral infection cycle.

Antigene oligonucleotides against genes involved in infectious diseases caused by viruses, bacteria, protozoa, fungi, parasites, etc., may be used in combinations. For instance, a cocktail of several different antigene oligonucleotides, directed against different regions of the same gene, may be administered simultaneously or separately. Also, combinations of antigene oligonucleotides specific for different genes, such as for instance the HBV P, S, and C gene, may be administered simultaneously or separately. Antigene oligonucleotides may also be administered in combination with other antiviral drugs, antibiotics, etc.

In the practice of the present invention, target genes may be single-stranded or double-stranded DNA or RNA. It is understood that the target to which the antigene oligonucleotides of the invention are directed include allelic forms of the targeted gene and the corresponding mRNAs including splice variants. There is substantial guidance in the literature for selecting particular sequences for antigene oligonucleotides given a knowledge of the sequence of the target polynucleotide. Preferred mRNA targets include the 5′ cap site, tRNA primer binding site, the initiation codon site, the mRNA donor splice site, and the mRNA acceptor splice site.

Where the target polynucleotide comprises a mRNA transcript, sequence complementary oligonucleotides can hybridize to any desired portion of the transcript. Such oligonucleotides are, in principle, effective for inhibiting translation, and capable of inducing the effects described herein. It is hypothesized that translation is most effectively inhibited by blocking the mRNA at a site at or near the initiation codon. Thus, oligonucleotides complementary to the 5′-region of mRNA transcript are preferred. Oligonucleotides complementary to the mRNA, including the initiation codon (the first codon at the 5′ end of the translated portion of the transcript), or codons adjacent to the initiation codon, are preferred.

A particular aspect of the invention is the use of modifications such as the use of LNA monomers to enhance the potency, specificity and duration of action and broaden the routes of administration of oligonucleotides comprised of current chemistries such as MOE, ANA, FANA, PS etc (ref: Recent advances in the medical chemistry of antisense oligonucleotide by Uhlman, Current Opinions in Drug Discovery & Development 2000 Vol 3 No 2). This can be achieved by substituting some of the monomers in the current oligonucleotides by LNA monomers. The LNA modified oligonucleotide may have a size similar to the parent compound or may be larger or preferably smaller. It is preferred that such LNA-modified oligonucleotides contain less than about 70%, more preferably less than about 60%, most preferably less than about 50% LNA monomers and that their sizes are between about 10 and 25 nucleotides, more preferably between about 12 and 20 nucleotides.

In another preferred embodiment, the antigene oligonucleotides are used to treat patients susceptible to or suffering from cancer. Genes which are over expressed in a cancer cell can be identified so that the antigene oligonucleotide selectively targets the cancer cell as opposed to normal cells. For example, Expressed Sequenced Tags (ESTs), can be used to identify nucleic acid molecules which are over expressed in a cancer cell [expressed sequence tag (EST) sequencing (Celis, et al., FEBS Lett., 2000, 480, 2-16; Larsson, et al., J. Biotechnol., 2000, 80, 143-57)]. ESTs from a variety of databases can be identified. For example, preferred databases include, for example, Online Mendelian Inheritance in Man (OMIM), the Cancer Genome Anatomy Project (CGAP), GenBank, EMBL, PIR, SWISS-PROT, and the like. OMIM, which is a database of genetic mutations associated with disease, was developed, in part, for the National Center for Biotechnology Information (NCBI). OMIM can be accessed through the world wide web of the Internet, at, for example, ncbi.nlm.nih.gov/Omim/. CGAP, which is an interdisciplinary program to establish the information and technological tools required to decipher the molecular anatomy of a cancer cell. CGAP can be accessed through the world wide web of the Internet, at, for example, ncbi.nlm.nih.gov/ncicgap/. Some of these databases may contain complete or partial nucleotide sequences. In addition, alternative transcript forms can also be selected from private genetic databases. Alternatively, nucleic acid molecules can be selected from available publications or can be determined especially for use in connection with the present invention.

Alternative transcript forms can be generated from individual ESTs which are within each of the databases by computer software which generates contiguous sequences. In another embodiment of the present invention, the nucleotide sequence of the target nucleic acid molecule is determined by assembling a plurality of overlapping ESTs. The EST database (dbEST), which is known and available to those skilled in the art, comprises approximately one million different human mRNA sequences comprising from about 500 to 1000 nucleotides, and various numbers of ESTs from a number of different organisms. dbEST can be accessed through the world wide web of the Internet, at, for example, ncbi.nlm.nih.gov/dbEST/index.html. These sequences are derived from a cloning strategy that uses cDNA expression clones for genome sequencing. ESTs have applications in the discovery of new genes, mapping of genomes, and identification of coding regions in genomic sequences. Another important feature of EST sequence information that is becoming rapidly available is tissue-specific gene expression data. This can be extremely useful in targeting selective gene(s) for therapeutic intervention. Since EST sequences are relatively short, they must be assembled in order to provide a complete sequence. Because every available clone is sequenced, it results in a number of overlapping regions being reported in the database. The end result is the elicitation of alternative transcript forms from, for example, normal cells and cancer cells.

Assembly of overlapping ESTs extended along both the 5′ and 3′ directions results in a full-length “virtual transcript.” The resultant virtual transcript may represent an already characterized nucleic acid or may be a novel nucleic acid with no known biological function. The Institute for Genomic Research (TIGR) Human Genome Index (HGI) database, which is known and available to those skilled in the art, contains a list of human transcripts. TIGR can be accessed through the world wide web of the Internet, at, for example, tigr.org. Transcripts can be generated in this manner using TIGR-Assembler, an engine to build virtual transcripts and which is known and available to those skilled in the art. TIGR-Assembler is a tool for assembling large sets of overlapping sequence data such as ESTs, BACs, or small genomes, and can be used to assemble eukaryotic or prokaryotic sequences. TIGR-Assembler is described in, for example, Sutton, et al., Genome Science & Tech., 1995, 1, 9-19, which is incorporated herein by reference in its entirety, and can be accessed through the file transfer program of the Internet, at, for example, tigr.org/pub/software/TIGR. assembler. In addition, GLAXO-MRC, which is known and available to those skilled in the art, is another protocol for constructing virtual transcripts. Identification of ESTs and generation of contiguous ESTs to form full length RNA molecules is described in detail in U.S. application Ser. No. 09/076,440, which is incorporated herein by reference in its entirety.

Genes which are overexpressed by cancer cells as compared to normal cells, for example, genes expressed at least 5 fold greater in pancreatic cancers compared to normal tissues can be identified. Gene expression can also be analyzed by Serial Analysis of Gene Expression (SAGE), which is based on the identification of and characterization of partial, defined sequences of transcripts corresponding to gene segments [SAGE (serial analysis of gene expression) (Madden, et al., Drug Discov. Today, 2000, 5, 415-425)]. These defined transcript sequence “tags” are markers for genes which are expressed in a cell, a tissue, or an extract, for example.

SAGE is based on several principles. First, a short nucleotide sequence tag (9 to 10 bp) contains sufficient information content to uniquely identify a transcript provided it is isolated from a defined position within the transcript. For example, a sequence as short as 9 bp can distinguish 262,144 transcripts given a random nucleotide distribution at the tag site, whereas estimates suggest that the human genome encodes about 80,000 to 200,000 transcripts (Fields, et al., Nature Genetics, 7:345 1994). The size of the tag can be shorter for lower eukaryotes or prokaryotes, for example, where the number of transcripts encoded by the genome is lower. For example, a tag as short as 6-7 bp may be sufficient for distinguishing transcripts in yeast.

Second, random dimerization of tags allows a procedure for reducing bias (caused by amplification and/or cloning). Third, concatenation of these short sequence tags allows the efficient analysis of transcripts in a serial manner by sequencing multiple tags within a single vector or clone. As with serial communication by computers, wherein information is transmitted as a continuous string of data, serial analysis of the sequence tags requires a means to establish the register and boundaries of each tag. The concept of deriving a defined tag from a sequence in accordance with the present invention is useful in matching tags of samples to a sequence database. In the preferred embodiment, a computer method is used to match a sample sequence with known sequences.

The tags used herein, uniquely identify genes. This is due to their length, and their specific location (3′) in a gene from which they are drawn. The full length genes can be identified by matching the tag to a gene data base member, or by using the tag sequences as probes to physically isolate previously unidentified genes from cDNA libraries. The methods by which genes are isolated from libraries using DNA probes are well known in the art. See, for example, Veculescu et al., Science 270: 484 (1995), and Sambrook et al. (1989), MOLECULAR CLONING: A LABORATORY MANUAL, 2nd ed. (Cold Spring Harbor Press, Cold Spring Harbor, N.Y.). Once a gene or transcript has been identified, either by matching to a data base entry, or by physically hybridizing to a cDNA molecule, the position of the hybridizing or matching region in the transcript can be determined. If the tag sequence is not in the 3′ end, immediately adjacent to the restriction enzyme used to generate the SAGE tags, then a spurious match may have been made. Confirmation of the identity of a SAGE tag can be made by comparing transcription levels of the tag to that of the identified gene in certain cell types.

Analysis of gene expression is not limited to the above methods but can include any method known in the art. All of these principles may be applied independently, in combination, or in combination with other known methods of sequence identification.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (reviewed in (Comb. Chem. High Throughput Screen, 2000, 3, 235-41)).

In yet another aspect, antigene oligonucleotides that selectively bind to variants of target genes are useful for treatment of cancer. For example, p53 mutants are well known in a variety of tumors. A “variant” is an alternative form of a gene. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

Sequence similarity searches can be performed manually or by using several available computer programs known to those skilled in the art. Preferably, Blast and Smith-Waterman algorithms, which are available and known to those skilled in the art, and the like can be used. Blast is NCBI's sequence similarity search tool designed to support analysis of nucleotide and protein sequence databases. Blast can be accessed through the world wide web of the Internet, at, for example, ncbi.nlm.nih.gov/BLAST/. The GCG Package provides a local version of Blast that can be used either with public domain databases or with any locally available searchable database. GCG Package v9.0 is a commercially available software package that contains over 100 interrelated software programs that enables analysis of sequences by editing, mapping, comparing and aligning them. Other programs included in the GCG Package include, for example, programs which facilitate RNA secondary structure predictions, nucleic acid fragment assembly, and evolutionary analysis. In addition, the most prominent genetic databases (GenBank, EMBL, PIR, and SWISS-PROT) are distributed along with the GCG Package and are fully accessible with the database searching and manipulation programs. GCG can be accessed through the Internet at, for example, http://www.gcg.com/. Fetch is a tool available in GCG that can get annotated GenBank records based on accession numbers and is similar to Entrez. Another sequence similarity search can be performed with GeneWorld and GeneThesaurus from Pangea. GeneWorld 2.5 is an automated, flexible, high-throughput application for analysis of polynucleotide and protein sequences. GeneWorld allows for automatic analysis and annotations of sequences. Like GCG, GeneWorld incorporates several tools for homology searching, gene finding, multiple sequence alignment, secondary structure prediction, and motif identification. GeneThesaurus 1.0™ is a sequence and annotation data subscription service providing information from multiple sources, providing a relational data model for public and local data.

Another alternative sequence similarity search can be performed, for example, by BlastParse. BlastParse is a PERL script running on a UNIX platform that automates the strategy described above. BlastParse takes a list of target accession numbers of interest and parses all the GenBank fields into “tab-delimited” text that can then be saved in a “relational database” format for easier search and analysis, which provides flexibility. The end result is a series of completely parsed GenBank records that can be easily sorted, filtered, and queried against, as well as an annotations-relational database.

In accordance with the invention, paralogs can be identified for designing the appropriate antigene oligonucleotide. Paralogs are genes within a species that occur due to gene duplication, but have evolved new functions, and are also referred to as isotypes.

The polynucleotides of this invention can be isolated using the technique described in the experimental section or replicated using PCR. The PCR technology is the subject matter of U.S. Pat. Nos. 4,683,195, 4,800,159, 4,754,065, and 4,683,202 and described in PCR: The Polymerase Chain Reaction (Mullis et al. eds, Birkhauser Press, Boston (1994)) and references cited therein. Alternatively, one of skill in the art can use the identified sequences and a commercial DNA synthesizer to replicate the DNA. Accordingly, this invention also provides a process for obtaining the polynucleotides of this invention by providing the linear sequence of the polynucleotide, nucleotides, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

In another preferred embodiment, the antigene locks can be used in treating diseases wherein immune cells are involved in the disease, such as autoimmune disease; hypersensitivity to allergans; organ rejection; inflammation; and the like. Examples of inflammation associated with conditions such as: adult respiratory distress syndrome (ARDS) or multiple organ injury syndromes secondary to septicemia or trauma; reperfusion injury of myocardial or other tissues; acute glomerulonephritis; reactive arthritis; dermatoses with acute inflammatory components; acute purulent meningitis or other central nervous system inflammatory disorders; thermal injury; hemodialysis; leukapheresis; ulcerative colitis; Crohn's disease; necrotizing enterocolitis; granulocyte transfusion associated syndromes; and cytokine-induced toxicity. Examples of autoimmune diseases include, but are not limited to psoriasis, Type I diabetes, Reynaud's syndrome, autoimmune thyroiditis, EAE, multiple sclerosis, rheumatoid arthritis and lupus erythematosus

As an example, Tables 7 through 10 lists a number of genes and protein products that may be modulated by antigene locks; table 7 (CD markers), table 8 (adhesion molecules) table 9 (chemokines and chemokine receptors), and table 10 (interleukins and their receptors). Also included are the genes encoding the immunoglobulin E (IgE) and the IgE-receptor (FcεRIα) as well as the genes for the other immunoglobulins, IgG₍₁₋₄₎, IgA₁, IgA₂, IgM, IgE, and IgD encoding free and membrane bound immunoglobulins and the genes encoding their corresponding receptors.

TABLE 7
CD markers

| | | | | |
|---|---|---|---|---|
| CD1a-d | CD30 | CD61 | CD91 | CD121 |
| CD2 | CD31 | CD62E | CDw92 | CD122 |
| CD3 | CD32 | CD62L | CD93 | CDw123 |
| CD4 | CD33 | CD62P | CD94 | CD124 |
| CD5 | CD34 | CD63 | CD95 | CDw125 |
| CD6 | CD35 | CD64 | CD96 | CD126 |
| CD7 | CD36 | CD65 | CD97 | CD127 |
| CD8 | CD37 | CD66a-e | CD98 | CDw128 |
| CD9 | CD38 | CD67 | CD99 | CD129 |
| CD10 | CD39 | CD68 | CD100 | CD130 |
| CD11a | CD40 | CD69 | CD101 | CDw131 |
| CD11b | CD41 | CD70 | CD102 | CD132 |
| CD11c | CD42a-d | CD71 | CD103 | CD133 |
| CDw12 | CD43 | CD72 | CD104 | CD134 |
| CD13 | CD44 | CD73 | CD105 | |
| CD14 | CD45 | CD74 | CD106 | |
| CD15 | CD46 | CDw75 | CD107a,b | |
| CD16 | CD47 | CDw76 | CDw08 | |
| CDw17 | CD48 | CD77 | CD109 | |
| CD18 | CD49a-f | CDw78 | CD110 | |
| CD19 | CD50 | CD79a,b | CD111 | |
| CD20 | CD51 | CD80 | CD112 | |
| CD21 | CD52 | CD81 | CD113 | |
| CD22 | CD53 | CD82 | CD114 | |
| CD23 | CD54 | CD83 | CD115 | |
| CD24 | CD55 | CDw84 | CD116 | |
| CD25 | CD56 | CD85 | CD117 | |
| CD26 | CD57 | CD86 | CD118 | |
| CD27 | CD58 | CD87 | CD119 | |
| CD28 | CD59 | CD88 | CD120a,b | |
| CD29 | CDw60 | CD89 | | |
| CD30 | | CD90 | | |

TABLE 8
Adhesion molecules

| | | | | |
|---|---|---|---|---|
| L-selectin | TCRγ/δ | BB-1 | Integrin α7 | Integrin α6 |
| P-selectin | CD28 | N-cadherin | Integrin α8 | Integrin β5 |
| E-selectin | LFA-3 | E-cadherin P- | IntegrinαV | Integrin αV |
| HNK-1 | PECAM-1 | cadherin | Integrin β2 | Integrin β6 |
| Sialyl- | VCAM-1 | Integrin β1 | Integrin αL | Integrin αV |
| Lewis X | ICAM-2 | Integrin α1 | IntegrinαM | Integrin β7 |
| | ICAM-3 | Integrin α2 | IntegrinαX | IntegrinαIEL |
| | Leukosialin | Integrin α3 | Integrin β3 | Integrin α4 |
| CD15 | HCAM | Integrin α4 | IntegrinαV | Integrin β8 |
| LFA-2 | CD45RO | Integrin α5 | IntegrinαIib | Integrin αV |
| CD22 | CD5 | Integrin α6 | | |
| ICAM-1 | HPCA-2 | | Integrin β4 | |
| N-CAM | | | | |
| Ng-CAM | | | | |
| TCRα/β | | | | |

TABLE 9
Chemokines and Chemokine receptors

| C-X-C | | | | Chemokine |
|---|---|---|---|---|
| chemokines | C-C chemokines | | C chemokines | Receptors |
| IL-8 | MCAF/MCP-1 | ABCD-1 | Lymphotactin | CCR1 |
| NAP-2 | MIP-1 α,β | LMC | | CCR2 |
| GRO/MGSA | RANTES | AMAC-1 | | CCR3 |
| γ IP-10 | I-309 | NCC-4 | | CCR4 |
| ENA-78 | CCF18 | LKN-1 | | CCR5 |
| SDF-1 | SLC | STCP-1 | | CCR6 |
| I-TAC | TARC | TECK | | CCR7 |
| LIX | PARC | EST | | CCR8 |
| SCYB9 | LARC | MDC | | CXCR1 |
| B cell-attracting | EBI 1 | Eotaxin | | CXCR2 |
| chemokine 1 | HCC-1 | | | CXCR3 |
| | HCC-4 | | | CXCR4 |
| | | | | CXCR5 |
| | | | | CX₃CR |

TABLE 10
Interleukins and their receptors

| | | | | |
|---|---|---|---|---|
| G-CSF | IL-2 Rα | IL-8 | IL-16 | TGF-β1 |
| G-CSF R | IL-2 Rβ | IL-9 | IL-17 | TGF-β1,2 |
| GM-CSF | IL-2 Rγ | IL-9 R | IL-18 | TGF-β2 |
| IFN-γ | IL-3 | IL-10 | PDGF | TGF-β3 |
| IGF-I | IL-3 Rα | IL-10 R | PDGF A Chain | TGF-β5 |
| IGF-I R | IL-4 | IL-11 | PDGF-AA | LAP TGF-β1 |
| IGF-II | IL-4 R | IL-11 R | PDGF-AB | Latent TGF-β1 |
| IL-1α | IL-5 | IL-12 | PDGF B Chain | TGF-β bpl |
| IL-1β | IL-5 Rα | IL-12 p40 | PDGF-BB | TGF-β RII |
| IL-1 RI | IL-6 | IL-12 p70 | PDGF Rα | TGF-β RIII |
| IL-1 RII | IL-6 R | IL-13 | PDGF Rβ | |
| IL-lrα | IL-7 | IL-13 Rα | TGF-α | |
| IL-2 | IL-7 R | IL-15 | TGF-β | |

It should be appreciated that in the above tables 7 through 10, an indicated gene means the gene and all currently known variants thereof, including the different mRNA transcripts to which the gene and its variants can give rise, and any further gene variants which may be elucidated. In general, however, such variants will have significant homology (sequence identity) to a sequence of a table above, e.g. a variant will have at least about 70 percent homology (sequence identity) to a sequence of the above tables 1-5, more typically at least about 75, 80, 85, 90, 95, 97, 98 or 99 homology (sequence identity) to a sequence of the above tables 7-10. Homology of a variant can be determined by any of a number of standard techniques such as a BLAST program.
Sequences for the genes listed in Tables 7-10 can be found in GenBank (http://www.ncbi.nlm.nih.gov/). The gene sequences may be genomic, cDNA or mRNA sequences. Preferred sequences are mammalian genes comprising the complete coding region and 5′ untranslated sequences. Particularly preferred are human cDNA sequences.

The methods of the invention can be used to screen for antigene lock polynucleotides that inhibit the functional expression of one or more genes that modulate immune related molecule expression. For example, the CD-18 family of molecules is important in cellular adhesion. Through the process of adhesion, lymphocytes are capable of continually monitoring an animal for the presence of foreign antigens. Although these processes are normally desirable, they are also the cause of organ transplant rejection, tissue graft rejection and many autoimmune diseases. Hence, antigene locks capable of attenuating or inhibiting cellular adhesion would be highly desirable in recipients of organ transplants (for example, kidney transplants), tissue grafts, or for autoimmune patients.

In another preferred embodiment, antigene lock oligonucleotides inhibit the expression of MHC molecules involved in organ transplantation or tissue grafting. For example, Class I and Class II molecules of the donor. Antigene locks inhibit the expression of these molecules thereby ameliorating an allograft reaction. Immune cells may be treated prior to the organ or tissue transplantation, administered at time of transplantation and/or any time thereafter, at times as may be determined by an attending physician. Antigene locks can be administered with or without immunosuppressive drug therapy.

In another preferred embodiment, antigene locks are used to treat individuals who W are hyper-responsive to an antigen such as an allergic individual. Antigene locks are designed to target V region genes known to produce IgE molecules specific for the allergan. IgE antibody specificity can be determined by routine immuno diagnostic techniques such as ELISA's, RIA's, PCR, Western Blots etc. From the amino acid sequence of the IgE molecules, the nucleic acid sequence can be deduced, using any of the database techniques described infra. Antigene locks are designed to bind to V region genes or any other part of a gene that makes encodes for the desired antibody, including rearranged and unrearranged immunoglobulin nucleic acid sequences.

In another preferred embodiment, antigene locks are designed to target suppressor molecules that suppress the expression of gene that is not suppressed in a normal individual. For example, suppressor molecules which inhibit cell-cycle dependent genes, inhibition of p53 gene, inhibition of genes coding for cell surface molecules (see tables 7-10), inhibition of caspases involved in apoptosis and the like.

Apoptosis is important clinically for several reasons. In the field of oncology, many of the clinically useful drugs kill tumor cells by inducing apoptosis. For example, cancer chemotherapeutic agents such as cisplatin, etoposide and taxol all induce apoptosis in target cells. In addition, a variety of pathological disease states can result from the failure of cells to undergo proper regulated apoptosis. For example, the failure to undergo apoptosis can lead to the pathological accumulation of self-reactive lymphocytes such as that occurring in many autoimmune diseases, and can also lead to the accumulation of virally infected cells and to the accumulation of hyperproliferative cells such as neoplastic or tumor cells. Antigene locks which target genes that encode for proteins that are capable of specifically inducing apoptosis would therefore be of therapeutic value in the treatment of these pathological diseases states.

In contrast, the inhibition of apoptosis is also of clinical importance. For example, cells are thought to die by apoptosis in the brain and heart following stroke and myocardial infarction, respectively. Moreover, the inappropriate activation of apoptosis can also contribute to a variety of other pathological disease states including, for example, acquired immunodeficiency syndrome (AIDS), neurodegenerative diseases and ischemic injuries. As apoptotic inducers are of benefit in the previously mentioned disease states, specific inhibitors of apoptosis would similarly be of therapeutic value in the treatment of these latter pathological disease states.

In a preferred embodiment, antigene locks target genes that prevent the normal expression or, if desired, over expression of genes that are of therapeutic interest as described above. As used herein, the term “overexpressing” when used in reference to the level of a gene expression is intended to mean an increased accumulation of the gene product in the overexpressing cells compared to their levels in counterpart normal cells. Overexpression can be achieved by natural biological phenomenon as well as by specific modifications as is the case with genetically engineered cells. Overexpression also includes the achievement of an increase in cell survival polypeptide by both endogenous or exogenous mechanisms. Overexpression by natural phenomenon can result by, for example, a mutation which increases expression, processing, transport, translation or stability of the RNA an well an mutations which result in increased stability or decreased degradation of the polypeptide. Such examples of increased expression levels are also examples of endogenous mechanisms of overexpression. A specific example of a natural biologic phenomenon which results in overexpression by exogenous mechanisms is the adjacent integration of a retrovirus or transposon. Overexpression by specific modification can be achieved by, for example, the use of antigene lock oligonucleotides described herein.

An antigene lock polynucleotide may be constructed in a number of different ways provided that it is capable of interfering with the expression of a target protein. The antigene lock polynucleotide generally will be substantially identical (although in a complementary orientation) to the target immune related molecule sequence. The minimal identity will typically be greater than about 80%, greater than about 90%, greater than about 95% or about 100% identical.

Preferred invention practice involves administering at least one of the foregoing antigene polynucleotides with a suitable nucleic acid delivery system. In one embodiment, that system includes a non-viral vector operably linked to the polynucleotide. Examples of such non-viral vectors include the polynucleoside alone or in combination with a suitable protein, polysaccharide or lipid formulation.

Additionally suitable nucleic acid delivery systems include viral vector, typically sequence from at least one of an adenovirus, adenovirus-associated virus (AAV), helper-dependent adenovirus, retrovirus, or hemagglutinating virus of Japan-liposome (HVJ) complex. Preferably, the viral vector comprises a strong eukaryotic promoter operably linked to the polynucleotide eg., a cytomeglovirus (CMV) promoter.

Additionally preferred vectors include viral vectors, fusion proteins and chemical conjugates. Retroviral vectors include moloney murine leukemia viruses and HIV-based viruses. One preferred HIV-based viral vector comprises at least two vectors wherein the gag and pol genes are from an HIV genome and the env gene is from another virus. DNA viral vectors are preferred. These vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.:90 7603 (1993); Geller, A. I., et al., Proc Natl. Acad. Sci. USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors may be an indication for some invention embodiments. The adenovirus vector results in a shorter term expression (eg., less than about a month) than adeno-associated virus, in some embodiments, may exhibit much longer expression. The particular vector chosen will depend upon the target cell and the condition being treated. The selection of appropriate promoters can readily be accomplished. Preferably, one would use a high expression promoter. An example of a suitable promoter is the 763-base-pair cytomegalovirus (CMV) promoter. The Rous sarcoma virus (RSV) (Davis, et al., Hum Gene Ther 4:151 (1993)) and MMT promoters may also be used. Certain proteins can expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication. See, Sambrook, et al., Molecular Cloning: A Laboratory Manual, Cold Spring Harbor Laboratory press, (1989). The plasmid vector may also include a selectable marker such as the β-lactamase gene for ampicillin resistance, provided that the marker polypeptide does not adversely effect the metabolism of the organism being treated. The cassette can also be bound to a nucleic acid binding moiety in a synthetic delivery system, such as the system disclosed in WO 95/22618.

If desired, the polynucleotides of the invention may also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Feigner and Holm, Bethesda Res. Lab. Focus, 11(2):21 (1989) and Maurer, R. A., Bethesda Res. Lab. Focus, 11(2):25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Another preferred antigene lock delivery method is to use single stranded DNA producing vectors which can produce the antigene locks intracellularly. See for example, Chen et al, BioTechniques, 34: 167-171 (2003), which is incorporated herein, by reference, in its entirety.

The effective dose of the nucleic acid will be a function of the particular expressed protein, the particular cardiac arrhythmia to be targeted, the patient and his or her clinical condition, weight, age, sex, etc.

One preferred delivery system is a recombinant viral vector that incorporates one or more of the polynucleotides therein, preferably about one polynucleotide. Preferably, the viral vector used in the invention methods has a pfu (plague forming units) of from about 10⁸ to about 5×10¹⁰ pfu. In embodiments in which the polynucleotide is to be administered with a non-viral vector, use of between from about 0.1 nanograms to about 4000 micrograms will often be useful eg., about 1 nanogram to about 100 micrograms.

In another preferred embodiment, the antigene locks are designed to target genes in a plant. The targeted gene may be an enzyme, a plant structural protein, a gene involved in pathogenesis, or an enzyme that is involved in the production of a non-proteinaceous part of the plant (i.e., a carbohydrate or lipid). By inhibiting enzymes at one or more points in a metabolic pathway or genes involved in pathogenesis, the effect may be enhanced: each activity will be affected and the effects may be magnified by targeting multiple different components. Metabolism may also be manipulated by inhibiting feedback control in the pathway or production of unwanted metabolic byproducts.

The present invention may be used to reduce crop destruction by other plant pathogens such as arachnids, insects, nematodes, protozoans, bacteria, or fungi. Some such plants and their pathogens are listed in Index of plant Diseases in the United States (U.S. Dept. of Agriculture Handbook No. 165, 1960); Distribution of Plant-Parasitic Nematode Species in North America (Society of Nematologists, 1985); and Fungi on Plants and Plant Products in the United States (American Phytopathological Society, 1989). Inhibition of target gene activity could be used to delay or prevent entry of an infectious disease organism into a particular developmental step (e.g., metamorphosis), if plant disease was associated with a particular stage of the pathogen's life cycle.

Introduction of the antigene locks into plants can be achieved in many ways. In the past decade, a number of techniques have been developed to transfer genes into plants (Potrykus, I., Annual Rev. Plant Physiol. Plant Mol. Biol. 42:205-225 (1991)). For example, chromosomally integrated transgenes have been expressed by a variety of promoters offering developmental control of gene expression. (Walden and Schell, Eur. J. Biochem. 192:563-576 (1990)). The most highly expressed genes in plants are encoded in plant RNA viral genomes. Many RNA viruses have gene expression levels or host ranges that make them useful for development as commercial vectors. (Ahlquist, P., and Pacha, R. F., Physiol. Plant. 79:163-167 (1990), Joshi, R. L., and Joshi, V., FEBS Lett. 281:1-8 (1991), Turpen, T. H., and Dawson, W. O., Amplification, movement and expression of genes in plants by viral-based vectors, Transgenic plants: fundamentals and applications (A. Hiatt, ed.), Marcel Dekker, Inc., New York, pp. 195-217. (1992)). For example, tobacco (Nicotiana tabacum) accumulates approximately 10 mg of tobacco mosaic tombamovirus (TMV) per gram of fresh-weight tissue 7-14 days after inoculation. TMV coat protein synthesis can represent 70% of the total cellular protein synthesis and can constitute 10% of the total leaf dry weight. A single specific RNA transcript can accumulate to 10% of the total leaf mRNA. This transcript level is over two orders of magnitude higher than the transcription level observed for chromosomally integrated genes using conventional plant genetic engineering technology. Most plant viruses contain genomes of plus sense RNA (messenger RNA polarity) (Zaitlin and Hull, Ann. Rev. Plant Physiol. 38:291-315 (1987)). Plus sense plant viruses are a very versatile class of viruses to develop as gene expression vectors since there are a large number of strains from some 22 plus sense viral groups which are compatible with a wide number of host plant species. (Martelli, G. P., Plant Disease 76:436 (1992)). In addition, an evolutionarily related RNA-dependent RNA polymerase is encoded by each of these strains. This enzyme is responsible for genome replication and mRNA synthesis resulting in some of the highest levels of gene expression known in plants.

In order to develop a plant virus as a gene vector, one must be able to manipulate molecular clones of viral genomes and retain the ability to generate infectious recombinants. The techniques required to genetically engineer RNA viruses have progressed rapidly. If the virus is an RNA virus, the virus is generally cloned as a cDNA and inserted into a plasmid. The plasmid is used to make all of the constructions. The genome of many plus sense RNA viruses can be manipulated as plasmid DNA copies and then transcribed in vitro to produce infectious RNA molecules (reviewed in Turpen and Dawson, Transgenic Plants, Fundamentals and Applications, Marcel Dekker, New York, pp. 195-217 (1992)).

The interaction of plants with viruses presents unique opportunities for the production of complex molecules as typified by the TMV/tobacco system (Dawson, W. O., Virology 186:359-367 (1992)). Extremely high levels of viral nucleic acids and/or proteins accumulate in infected cells in a brief period of time. The virus catalyzes rapid cell-to-cell movement of its genome throughout the plant, with no significant tissue tropism. The infection is maintained throughout the life of the plant. The plants are not significantly adversely affected by the viral infection since the virus causes little or no general cytotoxicity or specific suppression of host gene expression.

In another preferred embodiment, antigene locks are transduced into plants and/or plant cells using vectors such as, for example, a tumor inducing (Ti) plasmid or portion thereof found in the bacterium Agrobacterium. A portion of the Ti plasmid is transferred from the bacterium to plant cells when Agrobacterium infects plants and produces a crown gall tumor. This transferred DNA is hereinafter referred to as “transfer DNA” (T-DNA). The transfer DNA integrates into the plant chromosomal DNA and can be shown to express the genes carried in the transferred DNA under appropriate conditions. Another example, employs cauliflower mosaic virus (CaMV) DNA as a vector for introduction of desired antigene oligonucleotide sequences into plant cells. CaMV is a member of the caulimovirus group and contains a double-stranded DNA genome.

Another technique in which antigene locks can be transduced into plant cells is by called electroporation.

In another preferred embodiment, antigene lock oligonucleotides can be introduced into plants using viruses with a DNA genome. One group of plant viruses has been identified which contains a DNA, rather than RNA genome. T his group comprises the geminiviruses. Geminiviruses are plant viruses characterized by dumbbell-shaped twinned icosahedral particles (seen by electron micrograph). Some geminiviruses comprise two distinct circular single-stranded (ss) DNA genomes. Examples of such two genome or binary geminiviruses include tomato golden mosaic virus (TGMV) which has an “A” DNA and a “B” DNA, and Cassaya latent virus (CLV) which has a “1” DNA and a “2” DNA. Other geminiviruses such as maize streak virus (MSV) are believed to have a single circular ssDNA genome. Typically, two genome (binary) geminiviruses are transmitted by white flies, while single genome geminiviruses are transmitted by leaf hoppers. As a group, geminiviruses infect both monocotyledonous and dicotyledonous plants and thus exhibit a broad host range.

All geminivirus particles carry circular ssDNA. In infected plant cells, geminivirus DNA sequences have been detected as both ss and double-stranded (ds) DNA, in predominately circular form. In infected plants, such sequences exist in the plant cell nuclei, apparently as episomes, at several hundred copies per nuclei. Thus, unlike the transfer DNA (T-DNA) derived from the Ti plasmids of Agrobacterium, these geminivirus DNA sequences are not integrated into plant chromosomal DNA and generate multiple copies (e.g. more than 5) per infected cell. In infected plants, geminivirus particles released by an infected cell can infect other cells throughout the plant. In the two genome geminivirus systems such as TGMV, infectivity, replication and movement throughout the whole plant has thus far been shown to require the presence of both the A and B components. These vectors are described in U.S. Pat. No. 6,147,278 which is incorporated herein, in its entirety.

The practice of the present invention can suitably employ, unless otherwise indicated, conventional techniques of molecular biology and the like, which are within the skill of the art. Such techniques are explained fully in the literature. See e.g., Molecular Cloning: A Laboratory Manual, (J. Sambrook et al., Cold Spring Harbor Laboratory, Cold Spring Harbor, N.Y., 1989); Current Protocols in Molecular Biology (F. Ausubel et al. eds., 1987 and updated); Essential Molecular Biology (T. Brown ed., IRL Press 1991); Gene Expression Technology (Goeddel ed., Academic Press 1991); Methods for Cloning and Analysis of Eukaryotic Genes (A. Bothwell et al. eds., Bartlett Publ. 1990); Gene Transfer and Expression (M. Kriegler, Stockton Press 1990); Recombinant DNA Methodology (R. Wu et al. eds., Academic Press 1989); PCR: A Practical Approach (M. McPherson et al., IRL Press at Oxford University Press 1991); Cell Culture for Biochemists (R. Adams ed., Elsevier Science Publishers 1990); Gene Transfer Vectors for Mammalian Cells (J. Miller & M. Calos eds., 1987); Mammalian Cell Biotechnology (M. Butler ed., 1991); Animal Cell Culture (J. Pollard et al. eds., Humana Press 1990); Culture of Animal Cells, 2nd Ed. (R. Freshney et al. eds., Alan R. Liss 1987); Flow Cytometry and Sorting (M. Melamed et al. eds., Wiley-Liss 1990); the series Methods in Enzymology (Academic Press, Inc.); Techniques in Immunocytochemistry, (G. Bullock & P. Petrusz eds., Academic Press 1982, 1983, 1985, 1989); Handbook of Experimental Immunology, (D. Weir & C. Blackwell, eds.); Cellular and Molecular Immunology (A. Abbas et al., W.B. Saunders Co. 1991, 1994); Current Protocols in Immunology (J. Coligan et al. eds. 1991); the series Annual Review of Immunology; the series Advances in Immunology; Oligonucleotide Synthesis (M. Gait ed., 1984); and Animal Cell Culture (R. Freshney ed., IRL Press 1987).

In yet another aspect, the invention provides kits for targeting nucleic acid sequences of infectious disease organism, cancer, autoimmune diseases and the like. For example, the kits can be used to target any desired nucleic sequence, such as an HPV sequence. The kits of the invention have many applications. For example, the kits can be used to target and kill cells infected with a virus, or if the cells are at different stages of a tumor. In another example, the kits can be used to treat patients with a particular disease.

In one embodiment, a kit comprises: (a) an antigene lock that targets a desired nucleic acid sequence, and (b) instructions to administer to cells or an individual a therapeutically effective amount of antigene locks. In some embodiments, the kit may comprise pharmaceutically acceptable salts or solutions for administering the antigene locks.

Optionally, the kit can further comprise instructions for suitable operational parameters in the form of a label or a separate insert. For example, the kit may have standard instructions informing a physician or laboratory technician to prepare a dose of antigene lock. In another example, the kit may have instructions for treating a plant infected with virus, fungus and the like.

Optionally, the kit may further comprise a standard or control information so that a patient sample can be compared with the control information standard to determine if the test amount of an antigene lock is a therapeutic amount consistent with for example, a shrinking of a tumor or decrease in viral load in a patient.

All documents mentioned herein are incorporated herein by reference in their entirety.

The invention has been described in detail with reference to preferred embodiments thereof. However, it will be appreciated that those skilled in the art, upon consideration of this disclosure, may make modifications and improvements within the spirit and scope of the invention. The following non-limiting examples are illustrative of the invention.

### EXAMPLES

### Materials and Methods

### Primers and Antigene Lock Oligonucleotides.

All oligonucleotides were purchased from Research Genetics (Huntsville, Ala.). PCR primers were cartridge purified, while antigene lock oligonucleotides were gel purified. All antigene locks were phosphorylated at the 5′ terminal using ?-³²P-ATP or dATP as previously published. Control antigene locks were synthesized for each gene-specific antigene lock by counting the combined number of each base in the arms, and randomizing them. All other structural features were maintained by making the backbones complementary except for the mispairs corresponding to the terminal bases of the arms.

### Plasmids.

pUC19 was purchased from Life Technologies, Rockville, Md., and pSG5 from Stratagene, La Jolla, Calif. pUC19-ΔPL was constructed by digesting pUC19 with Sac I and Hind III restriction enzymes to remove the polylinker. The primers that contain an underlined Not I site, 5′-AGCTAGCAGCGGCCGCGACCAAGCT-3′ and 5′-TGGTCGCGGCCGCTGCT-3′, were mixed with the digested plasmid and incubated with T4 DNA ligase. Successful replacement was confirmed by DNA sequencing.

### Electrophoretic Mobility Shift Assay (EMSA).

³²P-labeled pUC19 antigene locks, sequence specific and control, (1.9 nM) were incubated with the various plasmids (1 μM) overnight at 37° C. in a reaction buffer containing 7 mM Tris-HCl, pH 7.6, 7 mM MgCl₂ and 50 mM NaCl in the presence and absence of T4 DNA Ligase (NEB, 400 U). Products were electrophoresed on a 1.5% agarose gel in 1×TBE buffer.

DNA Cycle Sequencing of Antigene Lock Bound pUC19.

The pUC19 sequence specific antigene lock (1.9 nM) were mixed with pUC19 (1 μM), denatured at 95° C. for 2 mins, in a reaction buffer containing 7 mM Tris-HCl, pH 7.6, 7 mM MgCl₂ and 50 mM NaCl, and allowed to cool to room temperature. The resulting reactants were then incubated in the presence and absence of T4 DNA ligase (400 U). The ligated and unligated samples were subjected to cycle sequencing using either primer A (5′-TACCGCACAGATGCGTAAGG-3′) or primer B (5′-ATGCAGCTGGCACGACAGGT-3′) and BigDye terminators (Applied Biosystems, Foster City, Calif.) per manufacturers instructions and analyzed on an ABI 3700.

### Bacterial Strains.

E. coli HB101 (supE44, hsd20(r_{B}-m_{B}-), recA13, ara-14, proA2, lacY1, galK2, rpsL20, xyl-5, mtl-1) and E. coli lac-8036 (ara, thi, trpE9777, Δpro-lac, F′-) were used in the chromosome targeting experiments. For the episome targeting experiments E. coli 8036/wt and E. coli 8036/+6 were used. E. coli 8036/wt is derived from 8036 with an F′ episome containing proA and proB genes in addition to the lac operon. Since the wt episome is phenotypically wild-type, despite containing the Iq mutation (lad upregulating promoter mutation) and the L8 mutation (lacZ promoter mutation abolishing responsiveness to Catabolite Activating Protein, CAP), it is designated “wt” for clarity. E. coli 8036/+6 is 8036 with an episome bearing a mutation in the +6 position 6 of the lacZ operator gene (T C).

Transformation of Bacteria with Antigene Locks.

Electro-competent bacteria were co-transformed with the antigene locks and the ampicillin plasmid pSG5 (molar ratio 9000:1) using a Biorad Micropulser™, program EC2 per manufacturer's instructions and plated onto ampicillin containing plates. Xgal plates containing tryptophan (55 μg/ml), thiamine (5 μg/ml), biotin (50.7 ng/ml), glucose (0.2%) and Xgal (50 μg/ml) were routinely used in the presence or absence of ampicillin (100 μg/ml) and/or proline (50 μg/ml).

### PCR Amplification of Episome Genes.

PCR amplifications were performed either directly on 25 microliters of washed saturated culture or following episome isolation using the Qiagen (Valencia, Calif.) plasmid isolation kit according to manufacturer's instructions. Reactions included 2.5 U Platinum Taq DNA polymerase (Life Technologies, Rockville, Md.) using 10 ng DNA template, 0.2 mM primers, 2 mM MgSO₄, 0.2 mM each dNTP, and 1×PCR buffer after denaturation for 30 sec at 94° C. followed by 35 cycles of 30 sec at 94° C., 30 sec at 55° C., and 60 sec at 68° C. in a 50 μl reaction per manufacturer's instructions. Primers used were: lacZ forward (−46 to −27, 5′-GGTATGGCATGATAGCGCCC-3) and reverse (1023 to 1036, 5′-GAATCGGCCAACGC-3′) yielding a 1082 bp product; proA forward (218-237, 5′-TGAAAGGCATTGCCGACGAT-3′) and reverse (649-628, 5′-CCGGGATTGTCGACTGTTCAC-3′) yielding a 431 bp product.

### Human Cervical Cancer Cell Lines.

To construct C33A/E7, the C33A parental cell line ((HPV E7 negative) was transfected with 2 μg of a full length HPV-16 E7 cDNA expression vector (pCMV-Neo-Bam-E7) using lipofectamine (Gibco, Grand Island, N.Y.) per manufacturers instructions and selected in 400 μg/ml G418 (Sigma). G418 resistant colonies were characterized by PCR, RT-PCR and sequencing using M13 tailed forward (5′-GTAAAACGACGGCCAGGCAACCAGAGACAACTGATCTCTACTG-3′) and reverse (5′-CAGGAAACAGCTATGACAGAACAGATGGGGCACACAATTCC-3′) PCR primers. Positive colonies were confirmed by western blotting for E7 using the mouse anti-E7 8C9 antibody (Zymed Labs, San Francisco, Calif.) at a 1:100 dilution per standard technique.

Transfection of Human Cell Lines with Antigene Locks.

All three human cervical cancer or mouse cell lines were plated into 6 well plates (300,000 to 700,000 cells/well) and transfected with 1.9 μg of antigene lock in triplicate, using lipofectamine 2000 (Invitrogen, Carlsbad, Calif.) according to manufacturers instructions. Prior to transfection, the antigene locks or their controls were randomly designated with a letter only. After 3 days recovery, cells from each well were subcultured, diluted 1:10,000 and plated in growth media in 96-well plates. After 4 weeks, colony growth was scored by phase microscopy in a double blind fashion. The percent cell reduction for each of the gene specific and control antigene locks was calculated relative to no lock controls. Percent reduction was compared pairwise using a paired t-test.

### Example 1

### Design of the Novel Gene Targeting Antigene Locks

Two major designs were incorporated into the in situ padlock probes for use as gene targeting antigene locks. First, rather than using a non-homologous backbone of the in situ padlock probes, both the backbone and the arms were constructed so that they were complementary to both target DNA strands, and therefore to each other (FIG. 1a, top left). In the cell, we hypothesize that an equilibrium exists between this closed inactive form and an active open form in which the antigene lock is denatured (FIG. 1a, top right). In the denatured form, the backbone and arms should, have the ability to bind to both strands of locally denatured target DNA, creating two DNA duplexes, using Watson and Crick pairing (FIG. 1a, bottom right). If such structures were ligated in the cell, both target DNA strands should be inextricably intertwined with the lock (FIG. 1a, bottom left). Such structures would be unable to denature as is required during either transcription or replication. We also hypothesized that they would likely be more resistant to single stranded cellular exonucleases, increasing the probability of reaching their DNA targets.

Since both arms bound to the backbone with the terminal bases juxtaposed, there was a possibility that a DNA ligase might act on these structures, thereby inactivating the antigene lock. Thus, mispairs were created between the terminal bases of the arms and the backbone (FIG. 1a, top left). This was accomplished by making the base changes in the backbone rather than in the arms so that the arms would maintain full complementarity to the target gene (FIG. 1a, bottom right). The antigene locks were tested for binding activity and DNA synthesis inhibition in vitro.

### Example 2

### Gene Targeting Antigene Locks Bind in a Sequence Specific Fashion and can be “Locked”

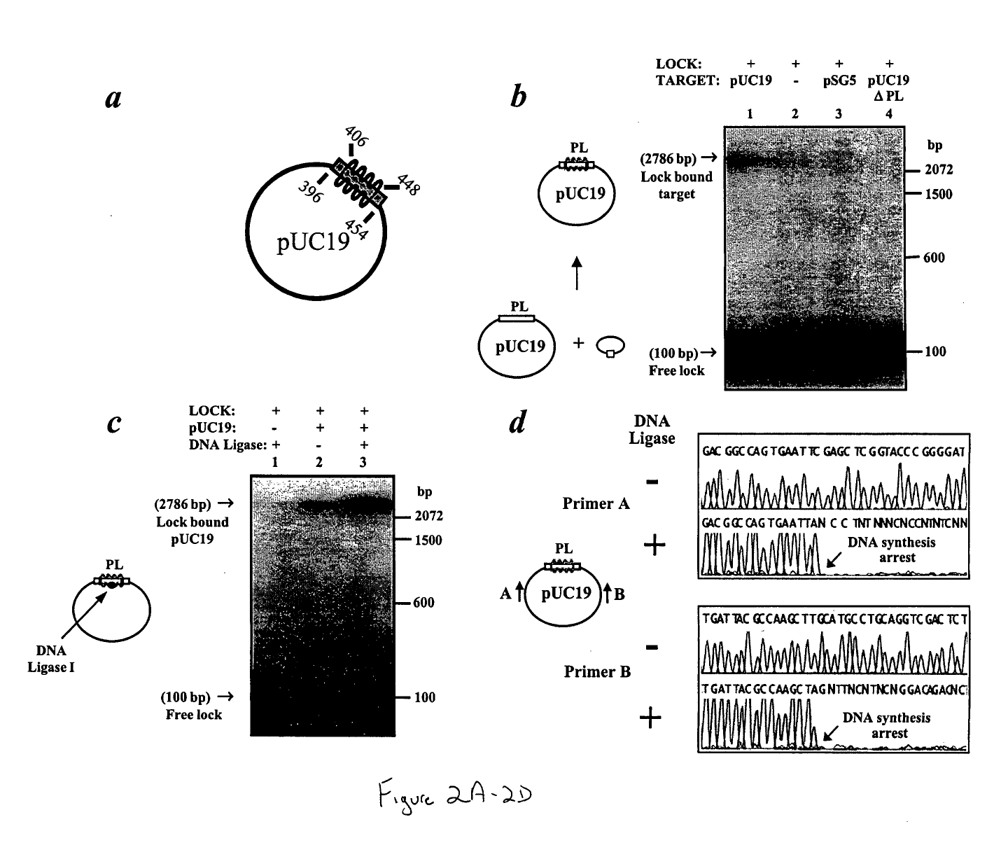

To initially determine if these novel antigene locks would bind specifically to target DNA, an antigene lock complementary to the pUC19 polylinker was synthesized to target the pUC19 polylinker in vitro (FIG. 2a). The ³²P end-labeled antigene lock was incubated with pUC19 plasmid containing the polylinker target at 37° C., overnight without any prior heat denaturation of either the plasmid or lock. Under these conditions the radioactively labeled antigene lock was shifted to a higher molecular mass, consistent with plasmid binding (FIG. 2b, lane 1). Specificity was demonstrated by the lack of a gel-shifted band when the pUC19 antigene lock was incubated alone without the plasmid (lane 2), with a plasmid that contains a different polylinker (pSG5, lane 3), or with a pUC19-derived plasmid in which the polylinker gene target had been deleted (pUC19-ΔPL, lane 4).

The antigene locks were designed with the hypothesis that ligation of the 5′ and 3′ terminal ends may be required for the structure to efficiently function as a replication or transcription inhibitor in the cell. With the addition of T4 DNA ligase to the in vitro reaction, at the end of the 37° C. overnight incubation with the plasmid, the antigene lock remain shifted and the amount of shifted form possibly increased (FIG. 2c, lane 3).

### Example 3

### Antigene Locks Inhibit In-Vitro DNA Synthesis

Having determined that the antigene lock was binding in a sequence dependent manner and that ligation was a factor possibly influencing the amount of binding, it was hypothesized that if bound and ligated as shown in FIG. 1 (lower left), then the two target DNA strands would not be capable of undergoing the normal strand denaturation required for DNA replication. To test this, the percent of plasmid with bound lock was maximized, and so the pUC19 plasmid was mixed with the antigene lock, heat denatured at 95° C. and cooled. Half of this reaction was then treated with T4 DNA ligase. The antigene lock bound plasmid samples, with or without DNA ligase treatment, were subjected to cycle sequencing to determine whether the presence of the ligated antigene lock would affect the ability of a DNA polymerase to synthesize DNA through the locked region.

As shown in FIG. 2d, using either of two sequencing primers (A and B) from both directions, sequence products end at the approximate position where the polymerase would be predicted to encounter the ligated antigene lock. Inhibition was observed only in the aliquot treated with DNA ligase. In the absence of DNA ligase, the DNA polymerase was able to sequence through this region, presumably by melting the unligated antigene lock from the target DNA strands during the denaturation phase of the cycle sequencing. This data indicated that specific binding and ligation of the antigene locks resulted in successful inhibition of in vitro DNA synthesis. However, while ligation is required to inhibit cycle sequencing based DNA synthesis in vitro, it is not indicative that it is necessarily required in cells.

### Example 4

### Antigene Lock Treatment of Episome Bearing Blue Bacteria Produce White Colonies

Since the antigene locks bind their gene target in a sequence specific manner in vitro and inhibit in vitro DNA synthesis, these locks were tested for their binding to a gene target inside a cell. A gene target was selected that was present in cells at low copy number, but would be reliably replicated and transmitted to daughter cells under normal conditions. Moreover, the DNA target was selected for ease of monitoring its presence or absence, and was non-essential for cell survival so that its loss could be detected in surviving cells. For these reasons, E. coli F′ episomes which are naturally occurring extra-chromosomal “big plasmids” that encode for pilus formation (designated “male”) and that allow bacteria to transmit the F′ to female bacteria (F′-) through conjugation 18. For genetic purposes, F′ elements can also contain other genes, such as the lac operon and the proA gene. The bacterial F′ episome chosen for study, designated “+6”, contains a mutation at the +6 position of the lac operator that prevents repressor binding such that the lacZ gene product, β-galactosidase, is constitutively expressed in these cells. The E. coli 8036 bacterial host, which harbors the F′, has a large deletion in its chromosome encompassing the lac operon and proA gene.

After determining that a radiolabeled antigene lock was stable from nuclease digestion, after transformation, for up to 8 hours in E. coli it was then determined whether the background spontaneous loss of the F′ episome was suitably low in the absence of selective pressure, by plating cells on Xgal (substrate for β-galactosidase) plates in the presence of exogenous proline. Only 1 white (Lac⁻) colony was observed among 528 blue (Lac⁺) colonies, confirming that spontaneous loss of the episome is a rare event.) Bacterial cells bearing this F′ (designated 8036/+6) were transformed with antigene locks directed towards either the F′ lacZ or proA genes. Control antigene locks were synthesized to contain the same number of each nucleotide as the sequence specific antigene locks, but with the sequence randomized (although the overall lock structure was maintained). The antigene and control locks were co-transformed with an ampicillin resistance encoding plasmid, pSG5, at a 9,000:1 lock:plasmid molar ratio to insure that all cells which had become ampicillin resistant, had also taken up antigene lock molecules. The transformed 8036/+6 cells were plated on Xgal plates containing ampicillin and proline. With both sequence specific locks, white colonies were produced on these plates at frequencies above those seen with the plasmid alone. When the control antigene locks were transformed into the same 8036/+6 cells, very few white colonies were produced. The frequencies that white colonies were produced with the antigene locks were significantly higher than their respective controls (p=0.01 and p=0.03).

### Example 5

### White Colonies are Due to Episome Loss

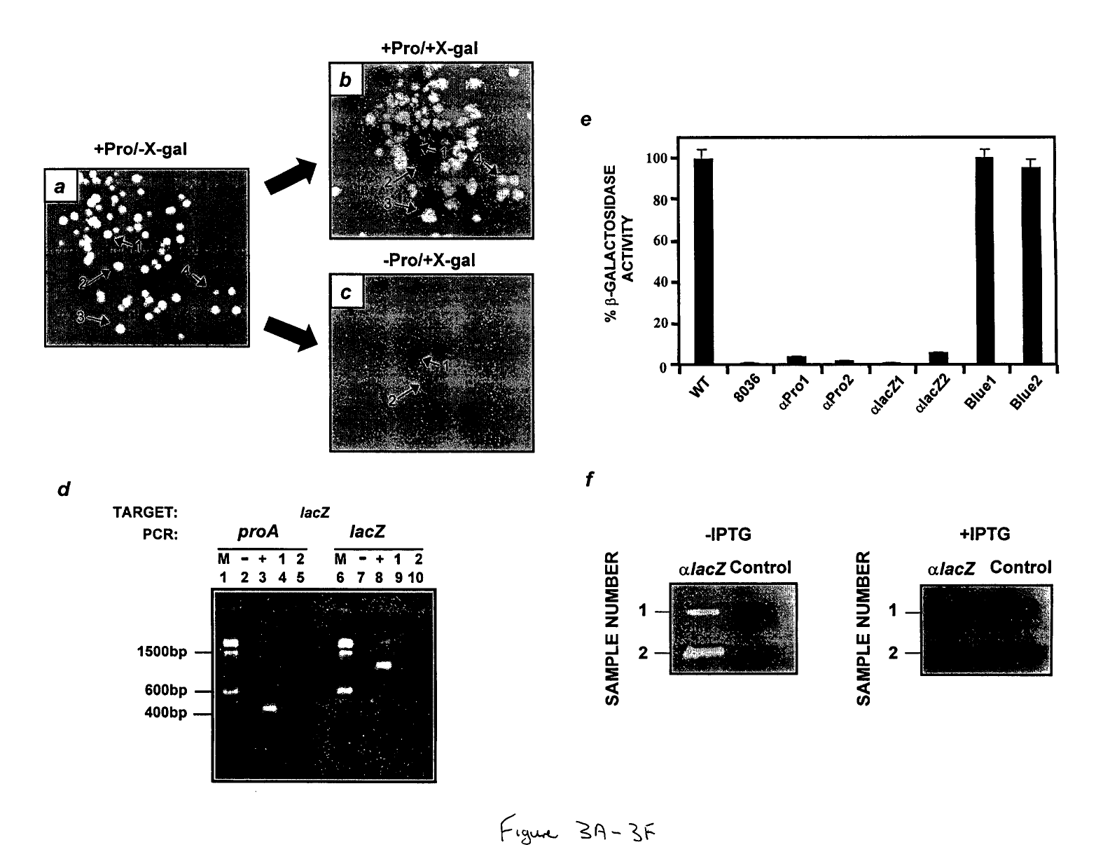

The production of white colonies suggested that either the F′ lacZ gene had been mutated or that the F′ episome had been degraded or eliminated from the cells. Experiments were conducted with the lacZ antigene lock but plated the cells on minimal media plates containing proline (FIG. 3a). These were then replicated onto Xgal plates with (FIG. 3b) and without (FIG. 3c) exogenous proline. In the presence of exogenous proline, both blue and white colonies survived, whereas in the absence of proline only the blue colonies survived (note the loss of colonies numbered 3 and 4). This suggested that treatment of the 8036/+6 cells with the sequence specific lacZ or proA antigene locks simultaneously resulted in loss of the ability to cleave Xgal (phenotypically LacZ⁻) and to synthesize proline (phenotypically Pro⁻). To genetically confirm these results, regions of both the F′ proA and lacZ genes from five white colonies, αlacZ1-5, were amplified using PCR. Neither gene could be amplified (data from 2 of the 5 colonies are shown in FIG. 3d, lanes 4, 5, 9 and 10), but were easily amplified from episomal DNA extracted from the blue, Pro⁺ colonies (lanes 3 and 8). PCR for three additional F′ genes, traK, repB and sopB, chosen because they were widely spaced around the episome, also did not amplify supporting the interpretation that the entire episome had been lost, rather than having suffered a localized deletion or mutation near the position of the lock. Similar results were observed using the proA antigene lock. Importantly, the total numbers of colonies were similar between the plasmid alone (21,302 colonies) and any of the sequence specific or control antigene lock treated samples (22,490 colonies, 106%). This suggests that the antigene locks were not non-specifically cytotoxic, but rather selectively caused the elimination or degradation of the targeted DNA structure.

To verify this further, both sets of white colonies, produced from the lacZ and proA antigene locks, and control blue colonies were examined for β-galactosidase activity. This correlated well with the results obtained from Xgal plating and PCR in that all the white colonies had minimal or no β-galactosidase activity (FIG. 3e). If the antigene lock treated bacteria had become F′ negative (and therefore become females), then they should be susceptible to conjugation with another episome. An F′ with a wildtype, inducible lac operon (designated 8036/wt) was chosen because it could easily determine whether they would exhibit the newly acquired phenotype. In contrast to the constitutive expression of the original +6 episome, the conjugated cells expressed β-galactosidase only when induced with IPTG, consistent with the wildtype phenotype and indicative of having taken up the new wild-type episome (FIG. 3f). When the phenotype, genotype and conjugation data are considered together, these results demonstrate that introduction of these sequence specific gene targeting antigene locks into these cells caused elimination of the target bearing F′ episome.

### Example 6

### Antigene Locks Kill Bacterial Cells when Directed to Chromosomal Targets

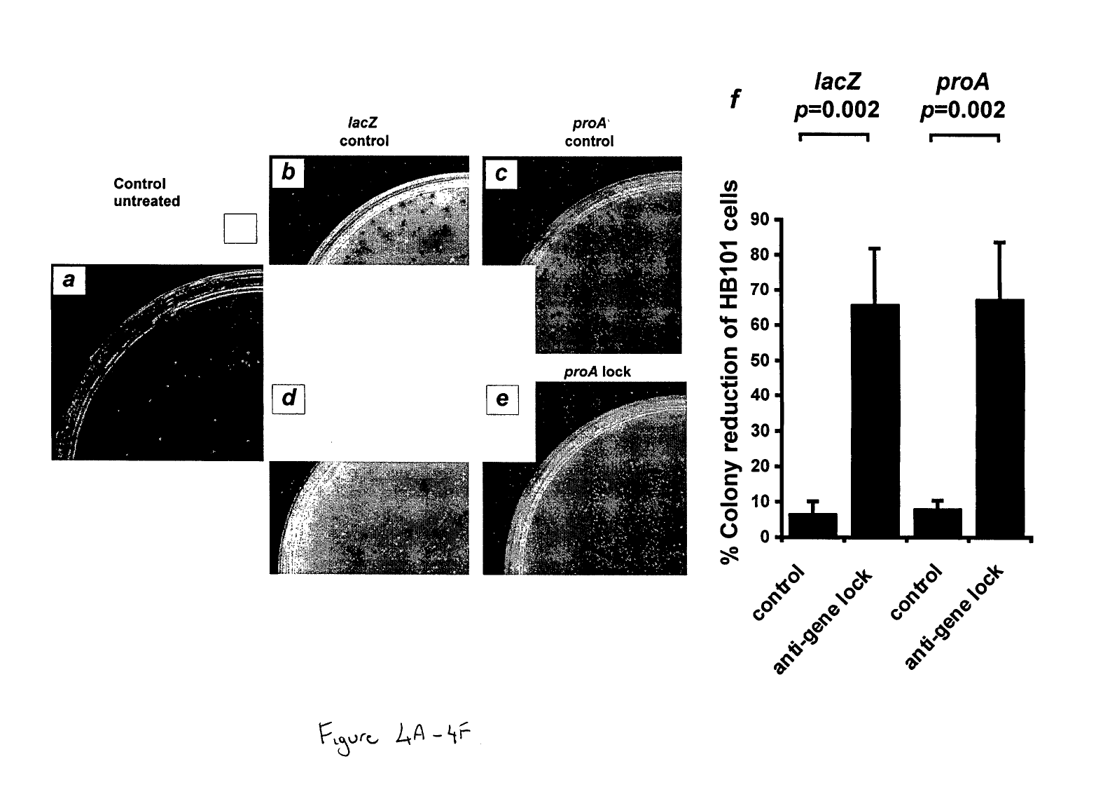

Since antigene locks caused the loss of an extra-chromosomal DNA element, it was determined whether the antigene locks would kill cells if their gene targets were present in the chromosome. To address this, initial work with bacterial cell mixtures was conducted, since the ratio of two different cell types (e.g. blue to white colonies) should be relatively consistent and not susceptible to tube-to-tube variation. To test whether antigene locks would be more toxic to a target bearing cell population, two bacterial cell populations were mixed together: E. coli HB101 (lacZ and proA wild-type E. coli) and the E. coli 8036 (in which both these genes have been deleted). In control experiments, where mixtures of these two cell types were either plated directly on Xgal/IPTG, or following transformation with the pSG5 plasmid, a relatively stable ratio of blue to white colonies was obtained (FIG. 4a). Following transformation of either the lacZ or proA directed antigene locks, about 65% reduction of blue colonies was achieved (FIGS. 4d, e and f). In contrast, the control randomized antigene locks exhibited relatively little reduction in the numbers of target-bearing blue colonies (FIGS. 4b, c and f, average 8% reduction). A comparison of results with each of the specific antigene locks with their control locks was highly significant (p=0.002 and p=0.002). The absolute number of white colonies remained relatively constant.

Since the two bacterial strains used in mix experiments were not isogenic, these findings were confirmed using only E. coli HB101 alone. Specific lacZ or proA antigene locks or their controls were co-transformed into HB101 and the percent colony reduction calculated relative to plasmid alone. The sequence specific antigene locks produced significant cell killing in the range of 30-35% (p=0.007 for lacZ and p=0.006 for proA). These results confirmed those conducted using the cell mixes, described infra.

### Example 7

### Do Antigene Locks Require Active Transcription to be Effective

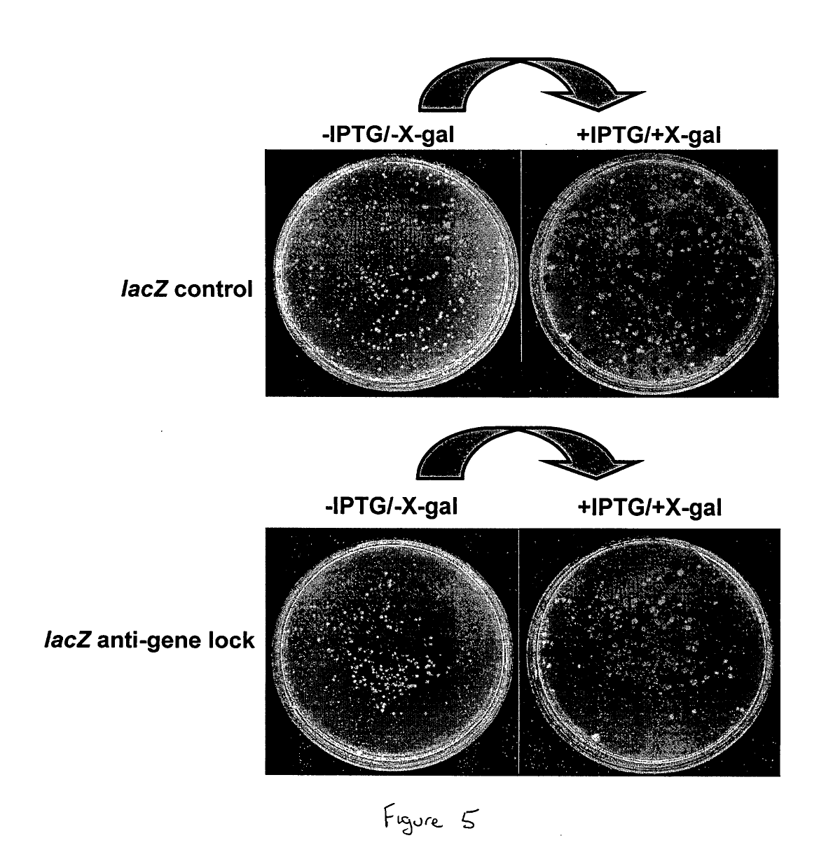

Successful gene targeting might require more accessible DNA and thus active transcription might be required for the antigene locks to be effective to killing bacterial cells. In the absence of IPTG (when lacZ transcription is repressed), the lacZ and control lacZ antigene locks were transformed into separate aliquots of a mixture of HB101/8036 cells and plated them on minimal media lacking IPTG. The resultant colonies were then replica plated onto Xgal plates with and without IPTG. Since the loss of blue colonies was still observed with the specific lacZ antigene lock (FIG. 5), it was concluded that active transcription is not a requirement for antigene locks to be effective in bacterial cells.

### Example 8

### Alu and HPV-E7 Antigene Locks Kill Human Cervical Cancer Cells

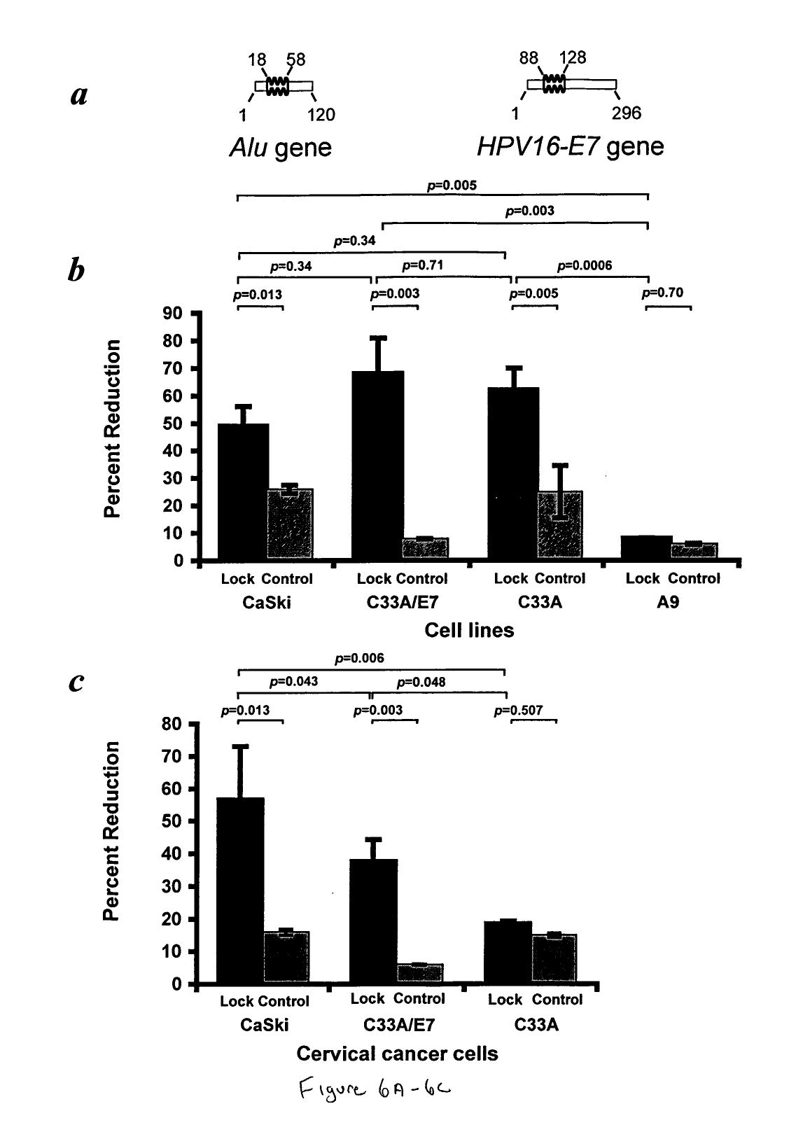

In demonstrating that the gene targeting antigene locks can specifically kill bacterial cells with chromosomal gene targets we hypothesized that they might kill human cells that contained unique gene targets also. Two targets were chosen as a proof of principle. The alu repeat sequence since there are hundreds of thousands of copies in the human genome (FIG. 6a). The human papilloma virus (HPV) E7 oncogene was chosen because it is completely foreign to the human genome, is present at a more biologically relevant copy number, and is found integrated in the human genome in ˜95% of human cervical cancers (FIG. 6a). Three human cervical cancer cell lines were tested, CaSki, C33A/E7 and C33A. While the alu repeat is present in all three of the cell lines, for HPV-16 E7, CaSki contains approx 300-500 copies per cell and serves as a positive control. C33A is one of the unusual cervical cancers that does not contain HPV elements, thereby serving as a negative control. C33A/E7 is a derivative of the C33A cell line in which the HPV-16 E7 oncogene was transfected.

Transfection of the alu sequence specific antigene lock into all three cell lines resulted in statistically significant levels of cell kill when compared to the alu control lock (FIG. 6b, CaSki, p=0.013, C33A/E7, p=0.003 and C33A, p=0.005). When transfected into mouse A9 cells, there was a negligible difference between the alu directed antigene lock and its control (p=0.70). The results showed that there were no significant differences between the effects of the alu specific antigene locks in any of the three human cell lines since all of them should contain roughly similar numbers of alu targets. Although some non-specific toxicity from the control lock was observed, a 2-8 fold increase in toxicity was seen with the alu antigene lock. However, since there are so many copies of the alu sequence, a more biologically relevant gene target was tested. Transfection of the E7 antigene lock into CaSki and C33A/E7 cell lines resulted in statistically significant levels of cell kill when compared to the E7 control lock in the same two cell lines (FIG. 6c, CaSki, p=0.013 and C33A/E7, p=0.003). The amount of cell kill with the specific E7 antigene lock was more significant in CaSki then C33A/E7 (p=0.043), presumably due to a larger number of E7 gene targets. As expected the amount of cell kill with the specific E7 antigene lock in both CaSki and C33A/E7 was more significant than in C33A (p=0.006 and p=0.048 respectively). It was noteworthy that the non-specific toxicity observed with the E7 control lock was relatively low, approximately 15%. In the control C33A line, which lacks the E7 gene target, the gene specific E7 antigene lock exhibited the same amount of non-specific toxicity as the E7 control lock that was the same as the E7 control locks in the other two cell lines.

### Example 8

### Antigene Locking Oligonucleotides

C33A cells are a cervical cancer cell line which does not contain any HPV elements. This cell line was transfected with a cDNA expression vector (pcDNA 3.1) containing the HPV-16 E7 oncogene. The transfected derivative line was selected in neomycin and characterized by PCR of genomic DNA for the presence of the transfected gene, in addition to RT-PCR and western blot for E7 expression. Antigene locks of total length 26 nucleotides or 86 nucleotides (predicted one turn and four turns of DNA respectively) were constructed as previously described. Oligonucleotides were synthesized by research genetics in a phosphorylated form and were gel purified. Table 11 and table 12 indicates the antigene oligonucleotides used:

TABLE 11
Antigene Locking Oligonucleotides

| Tar- | Size | |
|---|---|---|
| get | (bases) | Sequence¹ |
| HPV- | 26 | 5′-TGAAA-TTT-TTTCTACCTC-TTT-GAGGA-3′ |
| E7 | | (SEQ ID NO: 1) |
| HPV- | 86 | 5-TGAAATAGATGGTCCAGCTG-TTT- |
| E7 | | CAGCTGGACCATCTATTTCTACCTCCTCCTCTGAG |
| | | CTGTC-TTT-GACAGCTCAGAGGAGGAGGAGGA-3′ |
| | | (SEQ ID NO: 2) |
| β-gal | 26 | 5′-GTGAC-TTT-GTCAGCACGT-TTT-ACGTC-3′ |
| | | (SEQ ID NO: 3) |
| β-gal | 92 | 5′-GTGACTGGGAAAACCCTGGCG-TTT- |
| | | CGCCAGGGTTTTCCCAGGGTTTTCCCAGTCAGCACG |
| | | TTGTAAAACGACGGCCAG-TTT-CTGGCCGTCGTTT |
| | | TACAACGTC-3′ |
| | | (SEQ ID NO: 4) |

¹The base sequence is shown where the arms are completely complementary to the target. The backbone is complementary to the arms except for the position where the terminal bases of the arms would otherwise bind. Non-complementary bases were the same base as the terminal bases of the arms and were included to prevent self-litigation. The mispairs are underlined. Dashes separate the hinges (three thymidines) from the arms and the backbones.

TABLE 12
Anti-gene locks.

| | |
|---|---|
| pUC19^{a}: | 5′-AGTCGACCTGGAGGCATGCAA-TTT- |
| | TTGCATGCCTCCAGGTCGACAGTAGACGTTCCCC |
| | GCGTACCGA-TTT-TCGGTACGCGGGGATCCTCTAG-3′, |
| lacZ: | 5′-GTGACTGGGAAAACCCTGGCG-TTT- |
| | CGCCAGGGTTTTCCCAGTCAGCACGTTGTAAAAC |
| | GACGGCCAG-TTT-CTGGCCGTCGTTTTACAACGTC-3′. |
| lacZ | 5′-GGTTTAAGGGCGTACATCGAG-TTT- |
| control: | CTCGATGTACGCCCTTAAACGCCACTCAGGGAGC |
| | ATGGCGGAT-TTT-ATCCGCCATGCTCCCTGAGTGC-3′. |
| proA: | 5′-CCGAGGTTGCACACCTGACG-TTT- |
| | CGTCAGGTGTGCAACCTCGCCGATCCGGTGGGGCA |
| | GGTAA-TTT-TTACCTGCCCCACCGGATCC-3′. |
| proA | 5′-TGCGCACTATATGCGTTCGC-TTT- |
| control: | GCGAACGCATATAGTGCGCGGGGTGTGCGCGGCAT |
| | CGGGT-TTT-ACCCGATGCCGCGCACACCG-3′. |
| Alu: | 5′-GCTGGGATTACAGGCGTGAG-TTT- |
| | CTCACGCCTGTAATCCCAGGTCTTTGGGAGGCCGA |
| | GGTGG-TTT-CCACCTCGGCCTCCCAAAGT-3′ |
| Alu | 5′-TAGCCGAACCTAGTGGTACC-TTT- |
| control: | GGTACCACTAGGTTCGGCTGGAAGCTCCCCGGGTG |
| | AGCTG-TTT-CAGCTCACCCGGGGAGCTTG-3′ |
| HPV-E7: | 5′-TGAAATAGATGGTCCAGCTG-TTT- |
| | CAGCTGGACCATCTATTTCTACCTCCTCCTCTGAGCT |
| | GTC-TTT-GACAGCTCAGAGGAGGAGGA-3′ |
| HPV-E7 | 5′GATGATCATGAAGTAGGAAG-TTT- |
| control: | CTTCCTACTTCATGATCATGCCCCGCAGACTTCCGTT |
| | TCG-TTT-CGAAACGGAAGTCTGCGGGC-3′ |

^{a}Triple thymidines (separated by hyphens) represent the two hinges.

Human cells were plated into 6 well plates (C33A+E7 at 300,000 cells/well and the C33A control at 700,000 cells/well). They were transfected with 1.9 μg of antigene lock and 0.1 μg of pBABE-puro plasmid using lipofectamine 2000 according to the manufacturer's instructions. The cells were transfected in triplicate, and all the transfections were performed and analyzed double blinded. A lock of approximately equal length directed towards bacterial β-galactosidase was used as a negative control. After 48 hours, following transfection, cells were sub-cultured and plated into 96-well plates at 1:100 and 1:10,000 dilutions. Surviving colonies were counted 7-10 days after plating using phase microscopy. Table 13 shows the numbers of positive wells surviving.

TABLE 13
Cell Survival

| | | | No. of positive | |
|---|---|---|---|---|
| Cell Line | Lock | Plate number | wells | |
| C33A-E7 | E7-26 bases | 1 | | 6 |
| | | 2 | | 2 |
| | | 3 | | 1 |
| | | | Total = | 9 |
| C33A-E7 | β-gal-26 bases | 1 | | 15 |
| | | 2 | | 12 |
| | | 3 | | 9 |
| | | | Total = | 36 |
| C33A | E7-26 bases | 1 | | 23 |
| | | 2 | | 53 |
| | | 3 | | 31 |
| | | | Total = | 107 |
| C33A | β-gal-26 bases | 1 | | 46 |
| | | 2 | | 72 |
| | | 3 | | 70 |
| | | | Total = | 188 |
| C33A-E7 | E7-86 bases | 1 | | 5 |
| | | 2 | | 9 |
| | | 3 | | 8 |
| | | | Total = | 22 |
| C33A-E7 | β-ga1-92 bases | 1 | | 32 |
| | | 2 | | 25 |
| | | 3 | | 20 |
| | | | Total = | 77 |
| C33A | E7-86 bases | 1 | | 40 |
| | | 2 | | 51 |
| | | 3 | | 44 |
| | | | Total = | 135 |
| C33A | β-gal-92 | 1 | | 54 |
| | | 2 | | 84 |
| | | 3 | | 88 |
| | | | Total = | 226 |

While there was some apparent toxicity in control lines, a significantly larger amount is seen in the cells possessing the target. This corresponds to a percent cell kill of 75% for the 26 base E7 lock ((36−9)/36=75%) and 71% ((77−22)/77) for the 86 base E7 lock. Chi-squared analysis of results using the 26 base oligonucleotides is significant at p=0.032, and p=0.0048 for the 86 base locking oligonucleotides.

These data indicate that the antigene lock treatment of human cells results in the lack of the cells to produce clonal growth. This is evident in human and bacterial cells containing the target and where the locking oligonucleotide is directed towards this target, and most likely indicates that the cells were killed. This interpretation is consistent with the phase microscopic analyses performed shortly after transfection.

1-66. (canceled)

67. An antigene lock oligonucleotide comprising a single linear oligonucleotide strand consisting of, from 5′ to 3′: a left arm, a backbone, and a right arm, where the left and right arms are complementary to the backbone.

68. The antigene lock oligonucleotide of claim 67, wherein the arms interact with the backbone in an anti-parallel fashion thereby producing a dumbbell shaped structure.

69. The antigene lock oligonucleotide of claim 67, where the backbone interacts with one strand of a DNA target and the arms interact with the other strand of the DNA target.

70. The antigene lock oligonucleotide of claim 67, wherein the backbone comprises at least one mismatching base compared to at least one of the arms.

71. The antigene lock oligonucleotide of claim 67, wherein the backbone comprises at least one mismatching base compared to each of the arms.

72. The antigene lock oligonucleotide of claim 67, wherein the antigene lock hybridizes with a genomic target molecule.

73. The antigene lock oligonucleotide of claim 67, wherein the antigene lock hybridizes with episomal structures.

74. The antigene lock oligonucleotide of claim 67, wherein the antigene lock hybridizes to a DNA or an RNA target.

75. The antigene lock oligonucleotide of claim 67, wherein the antigene lock comprises molecules or oligonucleotide sequences comprising ligase activity.

76. The antigene lock oligonucleotide of claim 67, wherein the target nucleic acid molecule in a cell is expressed in a disease state or is a foreign nucleic acid molecule.

77. The antigene lock oligonucleotide of claim 76, wherein the disease state is cancer or an infection organism.

78. The antigene lock oligonucleotide of claim 77, wherein the infectious organism is selected from the group consisting of bacteria, protozoa, fungi, and virus.

79. The antigene lock oligonucleotide of claim 67, wherein the antigene oligonucleotide comprises from 8 to 200 base units.

80. The antigene lock oligonucleotide of claim 67, wherein the antigene oligonucleotide comprises modified base units.

81. The antigene lock oligonucleotide of claim 87, wherein the modified base units are selected from the group consisting of phosphorthiorate, methylphosphonate, peptide nucleic acids, and LNA molecules.

82. A method for inhibiting replication or transcription of a nucleic acid molecule indicative of a disease state, the method comprising:
targeting the nucleic acid molecule with the antigene lock oligonucleotide of claim 1; and
binding of the antigene lock oligonucleotide to the target nucleic acid molecule.

83. A method for selectively treating cells comprising an infectious disease organism, comprising:
administering to the cells the antigene lock oligonucleotide of claim 1, wherein the antigene lock oligonucleotide is complementary to a target nucleic acid molecule of an infectious disease organism.

84. A method of targeting cancer cells by administering to the cells the antigene lock oligonucleotide of claim 67, wherein the antigene lock oligonucleotide is complementary to a target nucleic acid molecule unique to the cancer cells.


SEQUENCE LISTING (27 sequences)

SEQ ID NO: 1 - DNA, 26 bp, Artificial Sequence
  Description of Artificial Sequence Synthetic oligonucleotide
tgaaattttttctacctctttgagga

SEQ ID NO: 2 - DNA, 89 bp, Artificial Sequence
  Description of Artificial Sequence Synthetic oligonucleotide
tgaaatagatggtccagctgtttcagctggaccatctatttctacctcctcctctgagctgtctttgacagctcagaggaggaggagga

SEQ ID NO: 3 - DNA, 26 bp, Artificial Sequence
  Description of Artificial Sequence Synthetic oligonucleotide
gtgactttgtcagcacgttttacgtc

SEQ ID NO: 4 - DNA, 103 bp, Artificial Sequence
  Description of Artificial Sequence Synthetic polynucleotide
gtgactgggaaaaccctggcgtttcgccagggttttcccagggttttcccagtcagcacgttgtaaaacgacggccagtttctggccgtcgttttacaacgtc

SEQ ID NO: 5 - DNA, 25 bp, Artificial Sequence
  Description of Artificial Sequence Synthetic primer
agctagcagcggccgcgaccaagct

SEQ ID NO: 6 - DNA, 17 bp, Artificial Sequence
  Description of Artificial Sequence Synthetic primer
tggtcgcggccgctgct

SEQ ID NO: 7 - DNA, 20 bp, Artificial Sequence
  Description of Artificial Sequence Synthetic primer
taccgcacagatgcgtaagg

SEQ ID NO: 8 - DNA, 20 bp, Artificial Sequence
  Description of Artificial Sequence Synthetic primer
atgcagctggcacgacaggt

SEQ ID NO: 9 - DNA, 20 bp, Artificial Sequence
  Description of Artificial Sequence Synthetic primer
ggtatggcatgatagcgccc

SEQ ID NO: 10 - DNA, 14 bp, Artificial Sequence
  Description of Artificial Sequence Synthetic primer
gaatcggccaacgc

SEQ ID NO: 11 - DNA, 20 bp, Artificial Sequence
  Description of Artificial Sequence Synthetic primer
tgaaaggcattgccgacgat

SEQ ID NO: 12 - DNA, 21 bp, Artificial Sequence
  Description of Artificial Sequence Synthetic primer
ccgggattgtcgactgttcac

SEQ ID NO: 13 - DNA, 43 bp, Artificial Sequence
  Description of Artificial Sequence Synthetic primer
gtaaaacgacggccaggcaaccagagacaactgatctctactg

SEQ ID NO: 14 - DNA, 41 bp, Artificial Sequence
  Description of Artificial Sequence Synthetic primer
caggaaacagctatgacagaacagatggggcacacaattcc

SEQ ID NO: 15 - DNA, 92 bp, Artificial Sequence
  Description of Artificial Sequence Synthetic oligonucleotide
agtcgacctggaggcatgcaatttttgcatgcctccaggtcgacagtagacgttccccgcgtaccgattttcggtacgcggggatcctctag

SEQ ID NO: 16 - DNA, 92 bp, Artificial Sequence
  Description of Artificial Sequence Synthetic oligonucleotide
gtgactgggaaaaccctggcgtttcgccagggttttcccagtcagcacgttgtaaaacgacggccagtttctggccgtcgttttacaacgtc

SEQ ID NO: 17 - DNA, 92 bp, Artificial Sequence
  Description of Artificial Sequence Synthetic oligonucleotide
ggtttaagggcgtacatcgagtttctcgatgtacgcccttaaacgccactcagggagcatggcggattttatccgccatgctccctgagtgc

SEQ ID NO: 18 - DNA, 86 bp, Artificial Sequence
  Description of Artificial Sequence Synthetic oligonucleotide
ccgaggttgcacacctgacgtttcgtcaggtgtgcaacctcgccgatccggtggggcaggtaatttttacctgccccaccggatcc

SEQ ID NO: 19 - DNA, 86 bp, Artificial Sequence
  Description of Artificial Sequence Synthetic oligonucleotide
tgcgcactatatgcgttcgctttgcgaacgcatatagtgcgcggggtgtgcgcggcatcgggttttacccgatgccgcgcacaccg

SEQ ID NO: 20 - DNA, 86 bp, Artificial Sequence
  Description of Artificial Sequence Synthetic oligonucleotide
gctgggattacaggcgtgagtttctcacgcctgtaatcccaggtctttgggaggccgaggtggtttccacctcggcctcccaaagt

SEQ ID NO: 21 - DNA, 86 bp, Artificial Sequence
  Description of Artificial Sequence Synthetic oligonucleotide
tagccgaacctagtggtacctttggtaccactaggttcggctggaagctccccgggtgagctgtttcagctcacccggggagcttg

SEQ ID NO: 22 - DNA, 86 bp, Artificial Sequence
  Description of Artificial Sequence Synthetic oligonucleotide
tgaaatagatggtccagctgtttcagctggaccatctatttctacctcctcctctgagctgtctttgacagctcagaggaggagga

SEQ ID NO: 23 - DNA, 86 bp, Artificial Sequence
  Description of Artificial Sequence Synthetic oligonucleotide
gatgatcatgaagtaggaagtttcttcctacttcatgatcatgccccgcagacttccgtttcgtttcgaaacggaagtctgcgggc

SEQ ID NO: 24 - DNA, 35 bp, Artificial Sequence
  Description of Artificial Sequence Synthetic oligonucleotide
gacggccagtgaattcgagctcggtacccggggat

SEQ ID NO: 25 - DNA, 36 bp, Artificial Sequence
  Description of Artificial Sequence Synthetic oligonucleotide
gacggccagtgaattancctntnnncnccntntcnn

SEQ ID NO: 26 - DNA, 37 bp, Artificial Sequence
  Description of Artificial Sequence Synthetic oligonucleotide
tgattacgccaagcttgcatgcctgcaggtcgactct

SEQ ID NO: 27 - DNA, 37 bp, Artificial Sequence
  Description of Artificial Sequence Synthetic oligonucleotide
tgattacgccaagctagnttncntncnggacagacnc